(19) United States
(12) Patent Application Publication
(10) Pub. No.: US 2026/0062731 A1
(43) Pub. Date: Mar. 5, 2026

(54) A LOOPABLE TRANSLATOR FOR THE SYNTHESIS OF REPETITIVE PROTEINS

(71) Applicant: The Johns Hopkins University, Baltimore, MD (US)

(72) Inventors: Stephen D. Fried, Baltimore, MD (US); Sea On Lee, Baltimore, MD (US)

(21) Appl. No.: 19/108,209

(22) PCT Filed: Sep. 8, 2023
(86) PCT No.: PCT/US2023/073741

§ 371 Date: Mar. 3, 2025

Related U.S. Application Data
(60) Provisional application No. 63/374,949, filed on Sep. 8, 2022.

(51) Int. Cl. C12P 21/02; C12N 15/10; C12N 15/85

(52) U.S. Cl. CPC C12P 21/02; C12N 15/1031; C12N 15/85; C12N 2800/107; C12N 2830/002

(57) ABSTRACT

The present disclosure is directed to a genetic construct for performing loopable translation, a kit comprising the genetic construct, and a method of producing biomaterials comprising a highly repetitive protein by use of the genetic construct.

### RELATED APPLICATION INFORMATION

This application claims priority to U.S. Application No. 63/374,949 filed on Sep. 8, 2022, the contents of which are herein incorporated by reference.

### SEQUENCE LISTING STATEMENT

The contents of the electronic sequence listing titled 40101_601_ST26.xml (Size: 113,918 bytes; and Date of Creation: Sep. 8, 2023) is herein incorporated by reference in its entirety.

### TECHNICAL FIELD

The present disclosure generally relates to a genetic construct for performing loopable translation, more specifically, to a genetic construct for auto-catalytically inducing RNA sequences to form a closed loop, enabling the creation of repetitive protein sequences.

### BACKGROUND

Proteins serve as the building blocks for functional, tunable materials across the tree of life. Spider silks (composed of spidroins¹⁻⁶), connective matrices (predominantly composed of collagens⁷⁻⁹). biofilms,¹⁰⁻¹³ and squid ring teeth (composed of SRT proteins¹⁴⁻¹⁶) are four examples of materials with distinct mechanical properties and biological functions, whose properties are genetically encoded through their respective protein sequences. Nevertheless, there remain many opportunities and challenges towards the capacity to synthetically recapitulate (or improve upon) the biological processes responsible for the creation of these materials.

Recently, there have been several successes in the design of novel globular cage-like protein materials, generally based on motifs that self-assemble through symmetry.¹⁷⁻²⁰ On the other hand, natural fibrous proteins typically consist of highly repetitive low-complexity regions within polypeptides with long chain lengths, and display self-assembly on many length scales, spanning from the nanometer (protein-protein interactions), micrometer (phase separation), to the millimeter (filamentization). Proteins of this type are generally less amenable to rational design, and also are typically challenging to express.

Despite the sequence and functional diversity of extant fibrous proteins attested in nature.¹⁻¹⁶ some unifying features of these proteins are the presence of long, repetitive, low-complexity regions. Pioneering work by Kaplan and coworkers^{1,6} demonstrated an approach to reproduce such a protein architecture in a recombinant expression vector. In their strategy, a short repetitive unit is auto-ligated into tandem repeats by availing of self-complementary sticky ends from two restriction enzymes.^{1,6} The judicious choice of two enzymes that create identical sticky ends but recognize distinct recognition sites enables for directionality and prevents the formation of inverse repeats.

More recently work by Demirel and coworkers appropriated rolling circle amplification (RCA)²¹ as an alternative strategy to generate tandem repeat proteins, an approach that has been used to express large SRT polypeptides (42 repeats; 1260 residues) in E. coli (See. FIG. 1B).¹⁴ Nevertheless, existing technologies to generate large repetitive proteins for recombinant expression in microbial hosts suffer from a number of major limitations: (i) highly repetitive DNA sequences suffer from genetic instability due to spontaneous recombination in microbial hosts; (ii) large plasmids containing tandem repeat proteins can be inconvenient for routine molecular biology manipulations (e.g., PCR, sequencing, and transformation); (iii) the metabolic cost to replicate and transcribe large DNA and mRNA molecules undermines the goal of redirecting resources toward the creation of proteins. To address these limitations, the present disclosure provides an alternative approach based on loopable translation.

### SUMMARY

In one embodiment, the present disclosure is directed to a genetic construct for performing loopable translation. More specifically, the present disclosure is directed to a genetic construct auto-catalytically inducing RNA sequences to form a closed loop and enabling the creation of repetitive protein sequences. In some aspects, the disclosure provides a genetic construct comprising in the following order: (i) a 3′ portion of a Group I self-splicing intron: (ii) a C-terminal portion of a gene of interest, wherein the C-terminal portion of the gene of interest comprises a 3′-exonic context sequence at its 5′ end; (iii) a ribosome binding site (RBS); (iv) a N-terminal portion of the gene of interest, wherein the N-terminal portion of the gene of interest comprises a 5′-exonic sequence at its 3′ end; and (v) a 5′ portion of the Group I self-splicing intron. In other aspects, the genetic construct further comprises an initiator codon (AUG) and a downstream box (DB) sequence after the ribosome binding site (RBS). In still other aspects, the genetic construct further comprises a TEV protease cleavage site at the end of the C-terminal portion of a gene of interest.

In some aspects of the genetic construct, the Group I self-splicing intron is a T4 bacteriophage thymidylate synthase (td) intron. The T4 bacteriophage thymidylate synthase (td) intron can be substituted with a Tetrahymena thermophila rRNA intron, Chlamydomonas reinhardii rRNA intron, or T4 bacteriophage sunY intron. The T4 bacteriophage sunY intron can be substituted with a Tetrahymena thermophila rRNA intron.

In other aspects of the genetic construct, the 3′-exonic context sequence functions as an internal guide sequence (IGS) for site-specific splicing. In still other aspects of the genetic construct, the 5′-exonic context sequence functions as an internal guide sequence (IGS) for site-specific splicing. In still further aspects of the genetic construct, the 5′-exon context sequence includes at least 1 codon, at least 2 codons, at least 3 codons, at least 4 codons, or at least 5 codons. In some aspects of the genetic construct, the 5′ exon context sequence includes at least one codon, GGU (glycine). In other aspects of the genetic construct, the 5′-exon context sequence does not include any codon, but in which the N-terminal portion of the gene of interest ends with GGU (glycine). In still other aspects of the genetic construct, the 3′-exon context sequence includes at least 1 codon, at least 2 codons, at least 3 codons, at least 4 codons, or at least 5 codons. The 3′ exon context sequence includes at least one codon is CUA (leucine).

In some aspects of the genetic construct, the gene of interest have a length of from 700 nucleotides to 1500 nucleotides. In other aspect of the genetic construct, the gene of interest is a gene that encodes green fluorescent protein (GFP). The GFP gene has the sequence of SEQ ID NO: 1. In still other aspect of the genetic construct, the N-terminal portion of the GFP gene comprises nucleotides 1 to 52 of SEQ ID NO: 1 and the C-terminal portion of the GFP gene comprises nucleotides 53 to 241 of SEQ ID NO:1.

In some aspects of the genetic construct, the gene of interest encodes a biofilm forming CsgA protein, dragline spidroin protein (major ampullate spidroin protein 1 (MaSp1)) with soluble end-domains, a Squid Ring Teeth (SRT) protein, a collagen, or an mRNA-vaccine.

In some aspects of the genetic construct, the Group I self-splicing intron requires the use of at least two co-factors. In other aspects, the co-factors are a divalent cation and a guanosine nucleoside. In some aspects, a guanosine is required because the intron uses a two-step mechanism that begins with the free nucleoside cleaving the phosphodiester linkage between the 5′-exon and the intron by prepending itself to the 5′-most nucleotides of the intron. In other aspect, a divalent cation is required to stabilize the tertiary fold of the catalytic core. In still other aspects, the divalent cation is a magnesium cation. In still further aspects of the genetic construct, the divalent cation is used in an amount from about 1 to about 30 mM to increase construct performance.

In certain aspects of the genetic construct, the construct is pBAD-IdTEVDB.

In some aspects, the genetic construct contains a mutation in a 36-base pair region covering the RBS, a RBS spacer, an initiator codon, or a DB for increasing translational efficiency and product yield. In other aspects of the genetic construct, the mutation is introduced using a modified error-prone PCR method. The mutation is C-15A, G4C or C-15A/G4C. In still other aspects of the genetic construct, the initiator codon is an initiator methionine.

In some embodiments, the present disclosure provides a kit comprising the genetic construct described herein and at least two co-factors. In some aspects of the kit, the co-factors are a divalent cation and a guanosine nucleoside. In some aspects, a guanosine is required because the intron uses a two-step mechanism that begins with the free nucleoside cleaving the phosphodiester linkage between the 5′-exon and the intron by prepending itself to the 5′-most nucleotides of the intron. In other aspects, a divalent cation is required to stabilize the tertiary fold of the catalytic core. In other aspects of the kit, the divalent cation is a magnesium cation.

In some embodiments, the present disclosure provides a method of producing biomaterials. The method comprises the step of transforming a host cell with the genetic construct described herein, adding at least two co-factors, and culturing the host cell at a temperature of about 25° C. to about 37° C. to produce a biomaterial. In some aspects of the method, the genetic construct further comprises an inducible promoter. In other aspects of the method, the inducible promoter is an Arabinose-inducible pBAD promoter or a GroES promoter. In still other aspects of the method, the host cell is a bacterial cell, a yeast cell, or a mammalian cell. In still further aspects of the method, the host is a bacterial cell. In some aspects of the method, the bacterial cell is an E. coli or a gram-positive bacterium. In other aspects of the method, the gram-positive bacterium is Bacillus subtilis. In another aspect of the method, the co-factors are a divalent cation and a guanosine nucleoside. In still another aspect, a guanosine is required because the intron uses a two-step mechanism that begins with the free nucleoside cleaving the phosphodiester linkage between the 5′-exon and the intron by prepending itself to the 5′-most nucleotides of the intron. In other aspect, a divalent cation is required to stabilize the tertiary fold of the catalytic core. In still another aspect of the method, the divalent cation is a magnesium cation. In still further aspects of the method, the magnesium cation is added to the culture in an amount of about 1 to about 30 mM. In some aspects of the method, the biomaterial is dragline silk comprising spidroins, a biofilm comprising curli proteins such as curli subunit A (CsgA), looped extracellular matrix (ECM) proteins such as fibronectin, laminin, collagen, reticulin, keratin, and elastin, squid ring teeth (SRT) proteins, globular cage-like protein nanomaterials or any combinations thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

FIG. 1 shows approaches to generate tandem repeat proteins. FIG. 1A shows that a plasmid encoding a monomeric unit is cut with two restriction enzymes to generate an insert. The original plasmid is cut with one of the two restriction enzymes to create self-complementary sticky ends, which allows ligation of the insert back into its original vector, creating a plasmid that encodes a tandem repeat. Upon ligation in a tail-to-head manner, the restriction site is destroyed, allowing directional cloning (inverse repeats can be removed by re-digesting). This process can be repeated to generate larger 2ⁿ multimers. FIG. 1B shows that a short segment of DNA encoding a monomeric unit is digested and circularized in vitro. The circular DNA serves as the template for rolling circle amplification and generates a mixture of concatemers with different lengths. The desired size of the repetitive sequences can be selected by gel extraction, digested, and cloned back into an expression vector. FIG. 1C shows that in loopable translation, a plasmid is created in which a segment of DNA encoding a monomeric unit is incorporated within a permuted self-splicing Group I intron (grey blocks). The plasmid is transformed into an expression strain, its gene is transcribed into RNA, which is circularized. The circular mRNA translates into a repetitive protein product through ribosome looping.

FIG. 2 shows the design of a loopable translator with a coupled fluorescence GFP reporter. FIG. 2A shows the sequence, secondary structure, and mechanism of the self-splicing Group I intron from the thymidylate synthase (td) gene of T4 bacteriophage. This intron catalyzes two consecutive, site-specific phosphoryl transfer reactions, which in the natural form, results in the splicing of the two flanking exons (orange). The 5′ splice site (marked with a red arrow) is selected by base-pairing between the 5′ exonic sequence (5′ Ex) and an internal guide sequence (IGS) at the beginning of the intron (P1), while the 3′ splice site (marked with an orange arrow) is selected by a short 2-bp stem formed between the 3′ exonic sequence (3′ Ex) and the edge of the P1 loop (termed P10, shown by two gray lines). An exogenous guanosine (red) is required to initiate the reaction and becomes prepended to the 5′-terminus of the intron sequence (black). 5′* and 3′* denote the termini of the RNA molecule in its natural form. In the construct, circular permutation in the ORF region of P6a results in two new termini (labeled 5′ and 3′), and a permuted GFP sequence (green) is incorporated between 3′* and 5′ *. In this reorganized topology, intron activity will result in circularization of the internal ‘exonic’ region.

FIG. 2B shows the design of a GFP fluorescence reporter system for RNA circularization. A plasmid is created (pBAD-tdTEVDB) in which a permuted super-folder GFP (sfGFP) gene is incorporated within the permuted intron. The GFP is split such that the N-terminal portion (residues 1-52) is placed downstream of the C-terminal portion (residues 53-241). In between these two coding regions is inserted a TEV protease site and a ribosome binding site (RBS) along with an enhancing downstream box (DB) in frame with the GFP coding sequence. Upon transcription of this RNA, intron folding, and RNA circularization, an mRNA is formed which is competent to recruit ribosomes and generate full-length GFP. Because polymeric GFP is found to have low fluorescence, this system is co-expressed with TEV protease, which can liberate fluorescent GFP monomers from the primary chain product. FIG. 2C shows that a negative control plasmid (pBAD-sfGFP₁₋₅₂) encodes the protein product that would form in the absence of circularization (with the same promoter, origin, and selectable marker as pBAD-tdTEVDB). A positive control plasmid (pBAD-sfGFP) encodes the protein product that would form upon circularization. Importantly, it differs from a wild-type sfGFP in that it has a 10-residue long ‘scar sequence’ in between residues 52 and 53, which result from exonic context sequences (represented as orange boxes) that were retained to ensure proper base-pairing with the IGSs. pBAD-tdTEVDB-STOP is identical to pBAD-tdTEVDB except that a stop codon is placed at the end of GFP. FIG. 2D shows that pBAD-tdTEVDB generates a fluorescence signal that is significantly higher than the background level (P<0.0001 by Student's t-test) but represents 4.2% of the signal of the corresponding positive control (n=3). Fluorescence measurements were conducted in biological triplicate.

FIG. 3 shows that high Mg and reduced temperature enhances loopable translation. FIG. 3A is a bar chart showing the levels of fluorescence of several constructs (negative control (−), positive control (+), and pBAD-tdTEVDB) expressed at 37° C. with varying concentrations of guanosine supplemented to the growth media during expression assays. Additional guanosine had no significant effect on the fluorescence signal generated by pBAD-tdTEVDB. FIG. 3B is a bar chart showing the levels of fluorescence of the same set of constructs expressed at 37° C. with varying concentrations of MgCl₂ supplemented to the growth media during expressions assays. Higher MgCl₂ concentrations had a beneficial effect; relative to 1 mM MgCl₂, fluorescence levels in 20 mM MgCl₂ were 1.2-fold higher (P-value=0.01). FIG. 3C is a bar chart showing the levels of fluorescence of the same set of constructs with varying MgCl₂ supplemented to the growth media, but with expressions carried out at 30° C. instead of 37° C. Lower temperature had a significantly beneficial effect on fluorescence signal (2.8-fold at 20 mM MgCl₂. P-value <0.0001). Moreover, at the lower temperature, the fluorescence signal significantly benefitted from high concentrations of MgCl₂ (a: 1.9-fold, P-value=0.0006). Under these conditions, the loopable translator generated 16% the fluorescence of the positive control. Fluorescence levels from pBAD-tdTEVDB-STOP (see FIG. 2C, labeled ‘STOP’) was slightly higher than the negative control (b: 1.05-fold, P-value=0.04), but much less than the loopable translator (c: 4.8-fold, P-value <0.0001). Fluorescence measurements were conducted in biological triplicate; statistical tests were conducted with Student's t-test.

FIG. 4 shows the verification of RNA circularization and polyGFP synthesis. FIG. 4A is an Anti-His western blot image showing the protein products of pBAD-sfGFP, pBAD-tdTEVDB, pBAD-tdTEVDB-STOP, and pBAD-tdTEVDB-mCherry that were expressed with and without the pRK793 plasmid (expressing TEV protease). pBAD-tdTEVDB in the absence of TEV protease generated proteins of high molecular weight. Expression in the presence of TEV protease generates a species with the molecular weight of monomeric GFP (25 kDa). FIG. 4B provides densitometry analysis showing average intensities relative to a positive control for three biological replicates of the Western blot. FIG. 4C shows construct maps of the positive control for a mCherry experiment (pBAD-sfGFP (52-DVFLGLPFNI)-mCherry), pBAD-tdTEVDB, and pBAD-tdTEVDB-mCherry. pBAD-tdTEVDB-mCherry was designed to form a larger mRNA loop compared to that of pBAD-tdTEVDB, in which GFP would be expressed only upon circularization, whereas mCherry would be expressed independently of circularization. FIG. 4D is a bar chart showing the levels of fluorescence from the constructs shown in FIG. 4C at 30° C. with varying concentrations of MgCl₂. The positive control shows the native difference in fluorescence between GFP and mCherry and serves as a normalization factor. With pBAD-tdTEVDB-mCherry, 25% of the target mRNA achieved circularization at 20 mM Mg²⁺. The larger mRNA loop was detrimental for circularization, leading to lower levels of GFP fluorescence compared to those of pBAD-tdTEVDB (P=0.0066 by Student's t-test).

FIG. 5 shows the minimal context requirements for a loopable translator. FIG. 5A shows the secondary structure of the loopable translator highlighting the 10-amino acid “scar sequence” that is incorporated into the loop because of the inclusion of 30 nt of exonic context retained from the natural td gene (15 nt from the original 5′ exon, and 15 nt from the original 3′ exon). Each codon triplet is color coded, and deletions of this context sequence are represented through their corresponding color blocks in bar charts in FIG. 5C-FIG. 5E. FIG. 5B shows the secondary structure that is formed when all 15 nt (coding for DVFLG) of the 5′ exonic context sequence are deleted. The nucleotides that correspond to residues 50-52 of GFP (green) replace the original 5′ exon to pair with the IGS. Nucleotides marked with * (in red) represent compensatory mutations in the P1 IGS (termed the modified P1*). FIG. 5C is a bar chart showing the level of fluorescence from a truncation series in which the 5′ context sequence was deleted one codon at a time. FIG. 5D is a bar chart showing the level of fluorescence from a truncation series in which the 3′ context sequence was deleted one codon at a time. FIG. 5E provides the minimal context requirements. Overall, the entire 5′ context sequence can be deleted, and all but the last 3 nucleotides of the 3′ context sequence (which form P10) can be deleted. Strengthening P10 (P10*) did not have a beneficial effect. All fluorescence measurements were conducted in biological triplicate. FIG. 5F shows the scar context sequences. In this case, the gene of interest is GFP, the blue codon (glycine) is not necessary.

FIG. 6 shows the directed evolution on the initiation sequence of loopable translator with minimum context. FIG. 6A shows that error-prone PCR was performed on a short 36-bp amplicon encoding the initiation region (ribosome binding site (RBS), spacer, initiator methionine, and a downstream box (DB)) of the GFP reporter gene. FIG. 6B shows the bar chart showing the fluorescence signals from the top performing constructs selected from the first round of directed evolution (n=3 biological replicates following primary screen). A construct with a point mutation (G4C) generated signal that is significantly higher than the wildtype (pBAD-tdTEVDB) (1.9-fold; P=0.0065). FIG. 6C provides a histogram showing the fluorescence signal of 372 constructs screened in the first round of directed evolution relative to the wildtype. FIG. 6D is a bar chart showing the fluorescence signal from the top performing constructs selected from the second round of directed evolution (n=3 biological replicates following primary screen). A construct with an additional mutation (C-15A/G4C) generated signal that was ca. 3-fold higher than the wildtype (a: P=0.0039) and ca. 2-fold higher than the single mutant (b: P=0.033). FIG. 6E is a histogram showing the fluorescence signal of 720 constructs screened in the second round of directed evolution relative to G4C. All statistical tests were conducted using Student's t-test.

FIG. 7 shows applying pBAD-tdTEVDB to producing spider silk spidroin. FIG. 7A shows the illustration of non-looped and looped constructs containing repetitive units of dragline silk. A repetitive unit with 36 amino acids was chosen as a monomeric unit. Six constructs were generated, comprising 8, 16, or 24 tandem repeats of a repetitive protein unit derived from major ampullate spidroin protein 1 (MaSp1), cloned into either a standard expression vector or the loopable translator. FIG. 7B shows an Anti-His Western blot image demonstrating the protein products of the six constructs. Apparently low molecular weights from the loopable translator could be due to high insolubility and/or instability of the resulting protein products.

FIG. 8 shows that a loopable translator enables long repetitive proteins to be efficiently synthesized in cells through a plug-and-play plasmid featuring a self-splicing intron in a permuted topology.

FIG. 9 shows comparison of several blots showing that Bacillus subtilis can generate high molecular weight protein products much more efficiently. pBAD-tdTEVDB is the original loopable translator before improvement. pBAD-tdTEVDB_C-15A/G4C is the loopable translator after context truncation and directed evolution. FIG. 9A shows that the translational efficiency of pBAD-tdTEVDB_C-15A/G4C significantly improves with the construct modifications. FIG. 9B shows that a loopable translator that is compatible with B. subtilis, pLIKE-td-TEVDB-C-15A_G4C, generates significantly more high molecular weight protein products compared to its counterpart in E. coli. Also, the loopable translator does not generate any monomeric byproduct in B. subtilis. FIG. 9C-D shows that in both E. coli and B. subtilis, the loopable translators operate well across diverse expression temperatures (25° C.-37° C.), though show better performance at 28° C. and 30° C.

FIG. 10 shows development of a coupled fluorescence for loopable translation. FIG. 10A shows bar chart showing the level of fluorescence of the controls, prototypes, and loopable translator. The original construct, td-FP-G-td (see Methods in the Examples section), generated signal that is only slightly higher than baseline (P value=0.0075 by Student's t-test). pBAD-tdTEVDB generated signal that is significantly higher than the baseline (P value <0.0001 by Student's t-test). FIG. 10B shows bar chart showing the relative performance of the same set of constructs compared to the positive control following background subtraction. pBAD-tdTEVDB generated significantly higher fluorescence compared to the prototype td-FP-G-td design (2.8-fold, P-value=0.0009 by Student's t-test). FIG. 10C shows constructs designed for the loopable translation.

FIG. 11 shows a cell growth curve. FIG. 11A shows that a negative control, a positive control, and pBAD-tdTEVDB were expressed in BL21+pRK793 and grown at 30° C. in a 96 well plate as OD₆₀₀ was measured in every 10 min over the course of 16h. BL21+pRK793 harboring each of the three constructs were inoculated in LB media supplemented with antibiotics (ampicillin and tetracycline), IPTG (0.1 mM), arabinose (0.2%), and 20 mM Mg²⁺ to staring ODs of 0.05. FIG. 11B is a bar chart showing the growth rates of the above constructs. No significant difference in growth rates was observed for the samples, indicating that there is no cell toxicity caused by loopable translation of pBAD-tdTEVDB.

FIG. 12 shows a Northern Blot imagine. FIG. 12A is a Northern blot image showing the RNA products generated from the negative control (pBAD-sfGFP 1-52) and pBAD-tdTEVDB. A biotinylated DNA probe (see Sequence Listing) was designed to anneal to RNA sequences corresponding to GFP residue 58 to 66 (present in the circularized loop generated by pBAD-tdTEVDB). Two ssRNA markers (ssRNA ladder (NEB); Low Range ssRNA ladder (NEB)) separated by electrophoresis under the identical condition to that of the Northern blot were used to estimate the size of the RNA bands. The linear RNA product prior to the autocatalytic cleavage is 1080 nt long, and the ribozymic intermediate is expected to be 900 nt long. Circular RNA is known to have significantly lower electrophoretic mobility, allowing assignment of high apparent-MW bands to circRNA. The lower of the two is tentatively assigned to a single supercoil. Low MW bands are presumed to be partial degradation products. FIG. 12B shows the densitometry of panel A, showing relative integrated intensities of the circularized and linear RNA products.

FIG. 13 highlights the constructs prepared for the truncation series in which codons are subsequently deleted one by one. For the 5′ exonic context truncation, three constructs were created: 1) deletion of D only; 2) deletion of DV; 3) deletion of the entire 5′ context sequence (DVFLG). For the 3′ exonic context truncation, five constructs were created: 1) deletion of I only; 2) deletion of NI; 3) deletion of FNI; 4) deletion of PFNI; 5) deletion of the entire 3′ context sequence (LPFNI). The construct shown in the bottom right (pBAD_tdTEVDB 5′td DVFLG+3′ PFNI Deletion) is the construct that generates the minimum scar upon circularization.

FIG. 14 shows a flow chart of several genetic constructs and how each construct was made.

FIG. 15 shows the sequence of pBAD-tdTEVDB before and after optimization with annotations shown in color.

The patent or application file contains at least one drawing executed in color. Copies of this patent or patent application publication with color drawings will be provided by the Office upon request and payment of the necessary fec.

### DETAILED DESCRIPTION

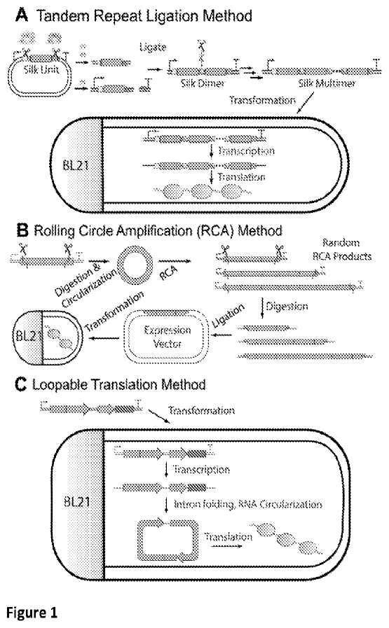

The present disclosure relates to a genetic construct for performing loopable translation as a loopable translator. More specifically, the present disclosure relates to a genetic construct auto-catalytically inducing RNA sequences to form a closed loop, enabling the creation of repetitive protein sequences. In contrast to previous approaches to generate tandem repeat proteins (such as those shown in FIG. 1A-B), the loopable translator of the present disclosure does not require large repetitive DNA constructs; rather, it uses a permuted group I self-splicing intron to circularize an mRNA transcript, thereby allowing ribosomes to translate a region of interest many times in succession as a single polypeptide chain. In some aspects, the present disclosure demonstrates a loopable translator combined with a fluorescence reporter system that can quantify the efficiency of circular translation in order to optimize its performance through rational considerations and by directed evolution. The present disclosure provides that such a system is a promising platform for the creation of fibrous proteins with repetitive sequence and hierarchical structure.

### I. Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. All publications, patent applications, patents and other references mentioned herein are incorporated by reference in their entirety. The materials, methods, and examples are illustrative only and not intended to be limiting.

As used herein, the term “about” or “approximately” means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

The terms “comprise(s),” “include(s).” “having.” “has,” “can.” “contain(s),” and variants thereof, as used herein, are intended to be open-ended transitional phrases, terms, or words that do not preclude the possibility of additional acts or structure. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It must be noted that as used herein and in the appended claims, the singular forms “a”, “an”, and “the” include plural references unless the context clearly dictates otherwise. All technical and scientific terms used herein have the same meaning.

As used herein, the term “genetic construct” refers to the assembly of various DNA sequences or RNA sequences transferred and integrated into a host genome.

As used herein, the phrase “group I self-splicing intron” refers to a group I intron that is structured into one or more self-splicing introns. These introns are widespread but sporadically distributed in nature, and are present in the genomes of some bacteria, mitochondria, chloroplasts, bacteriophages, and eukaryotic viruses, and bacteriophage. In one aspect, an example of a Group I self-splicing intron is a T4 bacteriophage thymidylate synthase (td) intron. In some aspects of the present disclosure, a T4 bacteriophage thymidylate synthase (td) intron can be substituted with a Tetrahymena thermophila rRNA intron, a Chlamydomonas reinhardii rRNA intron, or a T4 bacteriophage sun Y intron using routine techniques known in the art. In some other aspects, a T4 bacteriophage sun Y intron can be substituted with a Tetrahymena thermophila rRNA intron, using routine techniques known in the art.

As used herein, the phrase “internal guide sequence” refers to a polynucleotide sequence near the 5′-end of a group I intron that pairs with one or more sequences of an upstream exon as an intermediate of the self-splicing process.

As used herein, the phrase “TEV protease” refers to is a highly sequence-specific cysteine protease from Tobacco Etch Virus (TEV), which is used for the cleavage of fusion proteins and removal of tags from recombinant proteins in vitro or in vivo. In some aspects, a pRK793 plasmid can be used which contains a gene that encodes a soluble form of TEV protease (241 amino acids).

As used herein, the phrase “downstream box sequence (DB)” refers to a sequence reported to enhance translational efficiency. In some aspects, the downstream box sequence (DB) refers to a translational enhancer from Escherichia coli and/or bacteriophage mRNAs that are located just downstream of the initiation codon.

As used herein, the phrase “exonic sequence” refers to scar sequences in the junction site part of a Group I intron. Exonic sequences are included to ensure proper interactions with the internal guide sequences (IGS) of the Group I intron. In the present disclosure, the minimal exon context at the junction site is required for efficient intron activity.

As used herein, the phrase “modified error-prone PCR” refers to a specialized type of error-prone PCR a commonly employed approach in molecular biology, especially in directed evolution, to generate libraries of DNA molecules with broad mutational spectrums. In some aspects, modified error-prone PCR is a simple method that allows an arbitrarily high mutational load through iterative dilution/reamplification cycles. Specifically. the modified error-prone PCR method is based on a low-fidelity Mutazyme II DNA polymerase, iterating between dilution and reamplification, and touchdown PCR to suppress accumulation of incorrect product. In the present disclosure, this dilution and reamplification error-prone PCR method can be used to introduce mutations in a specific region (such as into a 36 base pair region), which is then cloned back into the genetic construct vector linearized with the reverse complement primers via In-Fusion cloning. To facilitate incorporation of mutations into a very short (36 base pair) amplicon a modified method which is described in Lee, S. O.; Fried, S. D. An Error-Prone PCR Method for Small Amplicons. Anal. Biochem. 2021, 628, 114266, the contents of which are herein incorporated by reference, can be used.

As used herein, the term “mammalian cell” refers to a type of eukaryotic cell having a nucleus and other cell structures that are bound by a distinct membrane. Eukaryotic cells are much more complex than bacterial cells. Mammalian cells include human mammalian cells and non-human mammalian cells. Non-human mammalian cells include, but are not limited to, Chinese hamster ovary (CHO) cells, mouse myeloma (e.g., NS0, SP2/0) cells, rat myeloma cell (e.g., YB2/0), and baby hamster kidney (BHK), etc. The human mammalian cells include, but are not limited to, human embryonic kidney cells (HEK293) and derivatives thereof, human retinal cells, HT-1080, and PER, etc.

As used herein, the term “bacterial cell” refers to a single-celled organism having a unique internal structure. Bacterial cells are prokaryotes, meaning they do not have organized nuclei or other membrane-bound organelles. In one aspect, a bacterium includes a gram-positive bacterium and a gram-negative bacterium. Examples of gram-negative bacteria include, but are not limited to, Pseudomonas, Klebsiella, Proteus, Salmonella, Providencia, Escherichia, Morganella, Aeromonas, and Citrobacter.

As used herein, the phrase “gram-positive bacterium” refers to a bacterium having one membrane. Examples of gram-positive bacterium include, but are not limited to, a coccus (spherical-shaped) and a bacillus (rod-shaped). The Coccus includes Staphylococcus (catalase positive) and Streptococcus (catalase negative). The Staphylococcus includes, but is not limited to, S. aureus, S. epidermidis, and S. saprophyticus. The streptococcus includes, but is not limited to, pyogenes, agalactiae, Enterococcus, E. faecalis, E. faecium, and pneumoniae. The Bacillus includes, but is not limited to, Corynebacterium, Clostridium, and Listeria. The Bacillus includes, but is not limited to, B. cereus, B. subtilis, B. anthracis, and B. thuringiensis.

As used herein, the phrase “yeast cell” refers to a eukaryotic, single-celled microorganism, such as fungi. A yeast cell includes, is not limited to, Saccharomyces cerevisiae, the genus Cryptococcus such as C. neoformans, the dimorphic fungus Candida albicans, etc.

As used herein, the term “biomaterial” refers to a substance that has been engineered to take a form which, alone or as part of a complex system, is used to direct, by control of interactions with components of living systems, the course of any therapeutic or diagnostic procedure. In certain aspects, a biomaterial is made of proteins produced by the loopable translator described herein, more specifically, by repetitive peptide chains or proteins. Examples of the biomaterial described herein include dragline silk comprising spidroins, a biofilm comprising curli proteins such as curli subunit A (CsgA), looped extracellular matrix (ECM) proteins such as fibronectin, laminin, collagen, reticulin, keratin, and elastin, squid ring teeth (SRT) proteins, globular cage-like protein nanomaterials, or any combination thereof.

### II. Genetic Construct for Performing Loopable Translation

In one embodiment, the present disclosure is directed to a genetic construct for performing loopable translation. In other embodiment, the present disclosure is directed to a genetic construct that circularizes mRNA in vivo by rearranging the topology of a group I self-splicing intron, thereby enabling “loopable” translation. In another embodiment, the genetic construct auto-catalytically induces RNA sequences to form a closed loop and enables the creation of repetitive protein sequences. The genetic constructs of the present disclosure are more stable and less susceptible to homologous recombination because the creation of repetitive DNA sequences is not required. In some aspects, the disclosure provides a genetic construct comprising in the following order: (i) a 3′ portion of a Group I self-splicing intron; (ii) a C-terminal portion of a gene of interest, wherein the C-terminal portion of the gene of interest comprises a 3′-exonic context sequence at its 5′ end; (iii) a ribosome binding site (RBS); (iv) a N-terminal portion of the gene of interest, wherein the N-terminal portion of the gene of interest comprises a 5′-exonic sequence at its 3′ end; and (v) a 5′ portion of the Group I self-splicing intron.

In another embodiment, the genetic construct further comprises an initiator codon (AUG) and a downstream box (DB) sequence after the ribosome binding site (RBS) and comprises in the following order: (i) a 3′ portion of a Group I self-splicing intron; (ii) a C-terminal portion of a gene of interest, wherein the C-terminal portion of the gene of interest comprises a 3′-exonic context sequence at its 5′ end; (iii) a ribosome binding site (RBS); (iv) a downstream box (DB) sequence; (v) a N-terminal portion of the gene of interest, wherein the N-terminal portion of the gene of interest comprises a 5′-exonic sequence at its 3′ end; and (vi) a 5′ portion of the Group I self-splicing intron.

In still other aspects, the genetic construct further comprises a TEV protease cleavage site at the end of the C-terminal portion of a gene of interest and comprises in the following order: (i) a 3′ portion of a Group I self-splicing intron; (ii) a C-terminal portion of a gene of interest, wherein the C-terminal portion of the gene of interest comprises a 3′-exonic context sequence at its 5′ end; (iii) a TEV protease cleavage site; (iv) a ribosome binding site (RBS); (v) a N-terminal portion of the gene of interest, wherein the N-terminal portion of the gene of interest comprises a 5′-exonic sequence at its 3′ end; and (vi) a 5′ portion of the Group I self-splicing intron.

In another aspect, the genetic construct further comprises an initiator codon (AUG) and a downstream box (DB) sequence after the ribosome binding site (RBS) and a TEV protease cleavage site at the end of the C-terminal portion of a gene of interest in the following order: (i) a 3′ portion of a Group I self-splicing intron; (ii) a C-terminal portion of a gene of interest, wherein the C-terminal portion of the gene of interest comprises a 3′-exonic context sequence at its 5′ end; (iii) a TEV protease cleavage site; (iv) a ribosome binding site (RBS); (v) a downstream box (DB) sequence; (vi) a N-terminal portion of the gene of interest, wherein the N-terminal portion of the gene of interest comprises a 5′-exonic sequence at its 3′ end; and (vii) a 5′ portion of the Group I self-splicing intron. The downstream box (DB) sequence is included for enhancing translational efficiency.

In some embodiments of the genetic construct, the Group I self-splicing intron is a T4 bacteriophage thymidylate synthase (td) intron. The T4 bacteriophage thymidylate synthase (td) intron can be substituted with a Tetrahymena thermophila rRNA intron using routine techniques known in the art. The T4 bacteriophage thymidylate synthase (td) intron can be substituted with a Chlamydomonas reinhardii rRNA intron using routine techniques known in the art. The T4 bacteriophage thymidylate synthase (td) intron can be substituted with a T4 bacteriophage sunY intron using routine techniques known in the art. The T4 bacteriophage sunY intron can be substituted with a Tetrahymena thermophila rRNA intron using routine techniques known the art. In one aspect of the genetic construct, the Group I self-splicing intron is circularly permutated at P6a.

In some embodiments of the genetic construct. the gene of interest has a length of from 700 nucleotides to 1500 nucleotides. In some embodiments of the genetic construct, the gene of interest have a length of from 500 nucleotides to 1500 nucleotides, from 600 nucleotides to 1500 nucleotides, from 700 nucleotides to 1500 nucleotides, from 800 nucleotides to 1500 nucleotides, from 900 nucleotides to 1500 nucleotides, from 1000 nucleotides to 1500 nucleotides, from 1100 nucleotides to 1500 nucleotides, from 1200 nucleotides to about 1500 nucleotides, from 1300 nucleotides to 1500 nucleotides, from 1400 nucleotides to 1500 nucleotides, from 500 nucleotides to 700 nucleotides, from 600 nucleotides to 700 nucleotides, from 700 nucleotides to 800 nucleotides, from 800 nucleotides to 900 nucleotides, from 900 nucleotides to 1000 nucleotides, from 1000 nucleotides to 1200 nucleotides, from 1000 nucleotides to 1300 nucleotides, or from 1000 nucleotides to 1500 nucleotides.

In one aspect, the gene of interest is a gene that encodes a green fluorescent protein (GFP). The GFP gene can have the sequence shown in SEQ ID NO:1. The N-terminal portion of the GFP gene can comprise nucleotides 1 to 52 of SEQ ID NO: 1 and the C-terminal portion of the GFP gene can comprise nucleotides 53 to 241 of SEQ ID NO:1. The GFP gene has been split into two pieces in a permuted fashion so that vectorial translation cannot produce a full-length GFP capable of fluorescing. Hence, GFP production is expressly dependent on translation from the circularized form of the mRNA; hence, GFP in this rearranged topology serves as a quantitative reporter for the circularization activity of the group I intron.

In other aspects, the gene of interest can encode a biofilm. Such biofilms can comprise curli proteins such as curli subunit A (CsgA). In another aspect, the gene of interest can encode a dragline spidroin protein with soluble end-domains, which is referred to as dragline silk. The dragline spidroin protein comprises spidroin I, spidroin II or combination thereof. In another aspect, the gene of interest encodes a Squid Ring Teeth (SRT) protein. In yet another aspect, the gene of interest encodes one or more looped extracellular matrix (ECM) proteins, such as fibronectin, laminin, collagen, reticulin, keratin, and elastin. In another yet another aspect, the gene of interest can encode an mRNA-based vaccine.

In some embodiments of the genetic construct. the C-terminal portion of the gene of interest (in the rearranged topology. N-terminal otherwise) includes a 3′-exonic context sequence at its 5′ end, and the N-terminal portion of the gene of interest (in the rearranged topology, C-terminal otherwise) includes a 5′-exonic context sequence at its 3′ end. The 3′-exonic context sequence and/or the 5′-exonic context sequence functions as an internal guide sequence (IGS) for site-specific splicing.

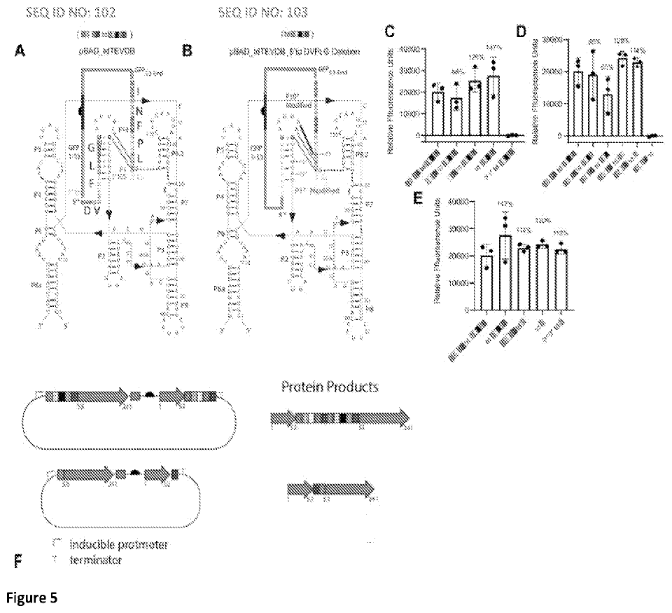

In some embodiments, the present disclosure provides the minimal exon context requirements for intron activity, such that the use of loopable translator system imposes minimal scar sequences at the junction site (See, FIG. 5E). In some aspects of the genetic construct. mRNA circularization can be accomplished with a scar as small as two amino acids at the junction region. In one aspect of the genetic construct, the 5′-exon context sequence includes at least 5 codons. In another aspect, the 5′-exon context sequence includes at least 4 codons. In another aspect, the 5′-exon context sequence includes at least 3 codons. In another aspect, the 5′-exon context sequence includes at least 2 codons. In another aspect, the 5′-exon context sequence includes at least 1 codon. In another aspect, the 5′-exon context sequence includes GAU, GUU, UUC, UUG, and GGU. In yet another aspect, the 5′-exon context sequence includes GUU, UUC, UUG, and GGU. In still a further aspect, the 5′-exon context sequence includes UUC, UUG, and GGU. In still yet a further aspect, the 5′-exon context sequence includes UUG and GGU. In yet still a further aspect, the 5′-exon context sequence includes GGU. In one aspect of the genetic construct, the 5′ exon context sequence includes at least one codon, GGU (glycine). In certain aspects, P1 (the beginning of the intron) requires (i) GGU (glycine) at the end of the N-terminal portion of the gene of interest (in the rearranged topology. C-terminal otherwise), and (ii) a total of 4 Watson-Crick (WC) base pairs in the P1 stem. Moreover, the IGS can be mutagenized to achieve the requisite number of WC base pairs at targeted positions. However, if the gene of the interest includes GGU (glycine) at the end of the N-terminal portion (in the rearranged topology, C-terminal otherwise), the 5′-exon context sequence does not need to include any codons. In some aspects, one example of a gene of interest that includes GGU is the GFP gene/super-folder GFP gene. Amino acid residues 50-52 of GFP are Thr-Thr-Gly, which are encoded by ACC/ACC/GGU, a sequence that conserves the three critical nucleotides (GGU) immediately prior to the intron. If the gene of the interest is a GFP gene, all 5 codons of its 5′-exon context sequence can be removed. In the case of the genetic construct having a GFP gene, the 5′-exon context sequence does not have to include any codons.

In other embodiment, the present disclosure identifies the minimal exon context requirements for intron activity, such that the use of loopable translator system imposes the minimal scar sequences required at the junction site (See, FIG. 5E). In one aspect of the genetic construct, the 3′-exon context sequence includes at least 5 codons. In another aspect. the 3′-exon context sequence includes at least 4 codons. In another aspect, the 3′-exon context sequence includes at least 3 codons. In another aspect, the 3′-exon context sequence includes at least 2 codons. In another aspect, the 3′-exon context sequence includes at least 1 codon. In one aspect, the 3′-exon context sequence includes CUA, CCG, UUU, AAU, and AUU. In another aspect, the 3′-exon context sequence includes CUA, CCG, UUU, and AAU. In another aspect, the 3′-exon context sequence includes CUA. CCG, and UUU. In another aspect, the 3′-exon context sequence includes CUA and CCG. In another aspect, the 3′-exon context sequence includes CUA. In some aspects, the 3′-exon context sequence should include at least one codon is CUA (leucine). In certain other aspects, P10 requires CUA (leucine) at the beginning of the C-terminal portion of the inserted gene. In some other aspects of the genetic construct, the minimal exon context requirements for intron activity combine the requirement for CUA immediately after the intron and GGU immediately before the intron. Therefore, the resulting protein concatemer contains a glycine and leucine as a scar in the middle of the sequence upon circularization.

In some embodiments of the genetic construct, the Group I self-splicing intron requires the use of at least two co-factors. In some aspects, the co-factors can be a divalent cation and/or a guanosine nucleoside. In other aspects, a guanosine is required because the intron uses a two-step mechanism that begins with a free nucleoside cleaving the phosphodiester linkage between the 5′-exon and the intron by prepending itself to the 5′-most nucleotides of the intron. In another aspect, a divalent cation is required to stabilize the tertiary fold of the catalytic core. In still another aspect, the divalent cation is a magnesium cation. In some aspects, the divalent cation can be used in an amount from about 1 to about 30 mM, from about 5 to about 30 mM, from about 10 to about 30 mM, from about 15 to about 30 mM, from about 16 to about 30 mM, from about 17 to about 30 mM, from about 18 to about 30 mM, from about 19 to about 30 mM, from about 20 to about 30 mM, from about 15 to about 25 mM, from about 17 to about 25 mM, from about 18 to about 25 mM, from about 20 to about 25 mM, from about 21 to about 25 mM, from about 22 to about 25 mM, from about 23 to about 25 mM to increase construct performance. In other aspects, the co-factors help with the folding and stability of the intron.

In some aspects, the genetic construct contains a mutation in a one or more of the 36-base pair region covering a ribosome binding site (RBS), a ribosome biding site (RBS) spacer. an initiator codon, or a downstream box (DB) for purposes of increasing translational efficiency and product yield. In another aspect, the genetic construct contains a mutation in one or more of a 36-base pair region covering the canonical ribosome binding site (RBS), the canonical RBS's spacer, an initiator codon, or a downstream box (DB) for purposes of increasing translational efficiency and product yield. In yet still another aspect, the canonical RBS is the Shine-Dalgarno (SD) sequence in prokaryotes or the Kozak sequence in vertebrates. In still further aspect of the genetic construct, the initiator codon is an initiator methionine.

In some aspects of the genetic construct, the mutation in the 36-base pair region is introduced using a modified error-prone PCR method, which is described in Lee, S. O.; Fried, S. D. An Error-Prone PCR Method for Small Amplicons. Anal. Biochem. 2021, 628, 114266, the contents of which are herein incorporated by reference. In other aspects, the modified error-prone PCR method is based on a low-fidelity Mutazyme II DNA polymerase, iterating between dilution and reamplification, and touchdown PCR to suppress accumulation of incorrect product. More specifically, this dilution and reamplification error-prone PCR method is used to introduce mutations into a 36 base pair region, which is then cloned back into the genetic construct vector linearized with the reverse complement primers via in-fusion cloning. In still further aspects, the mutation is C-15A, G4C or C-15A/G4C. In certain other aspects, the mutation is double mutant C-15A/G4C in the initiation region. The double mutant C-15A/G4C contains an additional mutation that extends the RBS sequence to include a run of 8 purines.

### III. a Genetic Construct for a Loopable Translator Coupled to a Fluorescence Reporter

In yet another embodiment, the present disclosure provides a loopable translator coupled to a fluorescence reporter. In other embodiment, the present disclosure provides a loopable translator coupled to a fluorescence reporter system that can quantify the efficiency of circular translation for optimized performance. In still other embodiments, the present disclosure provides a loopable translator coupled to a fluorescence reporter system that can quantify the expression from circular mRNA. In one aspect, a fluorescence-based assay can be used to probe the translational efficiency of circularized mRNAs. Such a system can be a platform for the creation of fibrous protein with a repetitive sequence and hierarchical structure.

In some embodiments, a genetic construct for a loopable translator coupled to a fluorescence reporter is designated as pBAD-td_{3′}-sfGFP_{53-end}-TEV-RBS-DB-sfGFP₁₋₅₂-td₅, which is abbreviated as pBAD-tdTEVDB. More specifically, pBAD-tdTEVDB comprises in the following order: (i) a 3′ portion of a Group I self-splicing intron; (ii) a C-terminal portion of a super-folder GFP, wherein the C-terminal portion of the super-folder GFP comprises a 3′-exonic context sequence at its 5′ end; (iii) a TEV protease cleavage site; (iv) a ribosome binding site (RBS); (v) a downstream box (DB) sequence; (vi) a N-terminal portion of a super-folder GFP, wherein the N-terminal portion of the super-folder GFP comprises a 5′-exonic sequence at its 3′ end; and (vii) a 5′ portion of the Group I self-splicing intron.

In one aspect of pBAD-tdTEVDB, the Group I self-splicing intron is a T4 bacteriophage thymidylate synthase (td) intron (herein after “T4 td intron”). In another aspect, the T4 td intron can be substituted with a Tetrahymena thermophila rRNA intron. In yet another aspect, the T4 td intron can be substituted with a Chlamydomonas reinhardii rRNA intron. In yet a further aspect, the T4 td intron can be substituted with a T4 bacteriophage sunY intron using routine techniques known in the art. In yet a further aspect, the T4 bacteriophage sun Y intron can be substituted with a Tetrahymena thermophila rRNA intron using routine techniques known in the art. In yet a further aspect, the T4 td intron is circularly permutated at Poa for circulation.

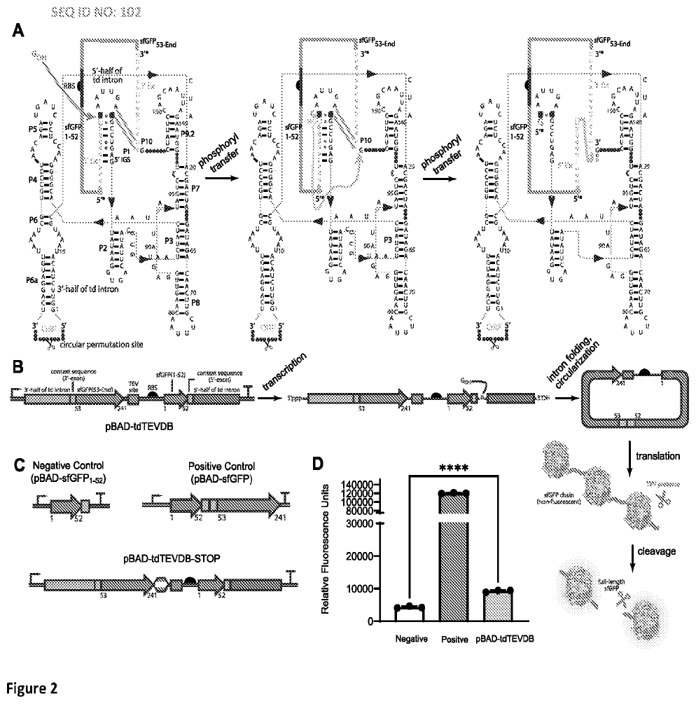

In one aspect of pBAD-tdTEVDB, a super-fold GFP gene encodes a GFP protein. The GFP concatemer chain/polymeric GFP is not fluorescent. but monomers of the GFP protein are fluorescent. In this system, circularized mRNA is initially translated into a GFP concatemer chain/polymeric GFP. Thus, the fluorescence assays require the activity of TEV protease (TEVP) to generate a readable signal. In certain aspects, in the present disclosure, the activity of TEV protease liberates GFP monomers from the chain allowing for fluorescence to be monitored in vivo (See, FIG. 2B). Thus, pBAD-tdTEVDB generates a single band at the expected molecular weight for monomeric GFP (25 kD) in the presence of TEV protease. For this reason, pRK793 plasmid containing a gene that encodes a soluble form of TEV protease (241 amino acids)^{29,30} is further incorporated into a host cell such as E. coli BL21 (DE21) to produce the TEV protease.

The GFP gene can have the sequence of SEQ ID NO:1. The N-terminal portion of the GFP gene can comprise nucleotides 1 to 52 of SEQ ID NO: 1 and the C-terminal portion of the GFP gene can comprise nucleotides 53 to 241 of SEQ ID NO: 1. In another aspect, the GFP gene is rearranged at residue 52, with the T4 td intron sequence following it, and the remainder of the GFP gene placed upstream of it. In still another aspect, exonic sequences are included to ensure proper interactions with the internal guide sequences (IGS) of the T4 td intron circularly permuted at P6a for circulation (See, FIG. 2A-B).

In still other embodiments of pBAD-tdTEVDB, the C-terminal portion of super-fold GFP gene (in the rearranged topology, otherwise the N-terminal) includes a 3′-exonic context sequence at its 5′ end, and the N-terminal portion of super-fold GFP gene (in the rearranged topology, otherwise the C-terminal) includes a 5′-exonic context sequence at its 3′ end. The 3′-exonic context sequence and/or the 5′-exonic context sequence functions as an internal guide sequence (IGS) for site-specific splicing.

In one embodiment of pBAD-tdTEVDB, the present disclosure identifies the minimal exon context requirements for intron activity, such that the use of loopable translator system imposes minimal scar sequences at the junction site (See, FIG. 5E). In one aspect of the genetic construct, the 5′-exon context sequence includes at least 5 codons. In another aspect. the 5′-exon context sequence includes at least 4 codons. In yet another aspect, the 5′-exon context sequence includes at least 3 codons. In still yet another aspect, the 5′-exon context sequence includes at least 2 codons. In yet another aspect, the 5′-exon context sequence includes at least 1 codon. In still yet a further aspect, the 5′-exon context sequence includes GAU, GUU, UUC, UUG, and GGU. In still yet another aspect, the 5′-exon context sequence includes GUU, UUC, UUG, and GGU. In still yet a further aspect, the 5′-exon context sequence includes UUC, UUG, and GGU. In still yet a further aspect, the 5′-exon context sequence includes UUG and GGU. In still yet a further aspect, the 5′-exon context sequence includes GGU. Specifically, in one aspect of the genetic construct, the 5′ exon context sequence includes at least one codon, GGU (glycine). In certain aspects, P1 (beginning of the intron) requires (i) GGU (glycine) at the end of the N-terminal portion of the gene of interest (in the rearranged topology, C-terminal otherwise), and (ii) a total of 4 Watson-Crick (WC) base pairs in the P1 stem. The IGS can be mutagenized to achieve the requisite numbers of WC base pairs at the targeted positions. However, if the gene of the interest includes GGU (glycine) at the end of the N-terminal portion, the 5′-exon context sequence does not need to include any codons. If the super-fold GFP gene includes GGU (glycine) at the end of the N-terminal portion, the 5′-exon context sequence does not need to include any codons. Therefore, all 5 codons of its 5′-exon context sequence can be removed.

In other embodiment of pBAD-tdTEVDB, the present disclosure identifies the minimal exon context requirements for intron activity, such that the use of loopable translator system impose minimal scar sequences at the junction site (See, FIG. 5E). In one aspect of the genetic construct, the 3′-exon context sequence includes at least 5 codons. In another aspect, the 3′-exon context sequence includes at least 4 codons. In another aspect, the 3′-exon context sequence includes at least 3 codons. In another aspect, the 3′-exon context sequence includes at least 2 codons. In another aspect, the 3′-exon context sequence includes at least 1 codon. In one aspect, the 3′-exon context sequence includes CUA, CCG, UUU, AAU, and AUU. In another aspect, the 3′-exon context sequence includes CUA, CCG, UUU, and AAU. In yet another aspect, the 3′-exon context sequence includes CUA, CCG, and UUU. In still yet another aspect, the 3′-exon context sequence includes CUA and CCG. In yet a further aspect, the 3′-exon context sequence includes CUA. The 3′-exon context sequence should include at least one codon is CUA (leucine). In certain aspects. P10 requires CUA (leucine) at the beginning of the C-terminal portion of the inserted gene (in the rearranged topology, N-terminal otherwise). In some aspects of the genetic construct, the minimal exon context requirements for intron activity are to combine the requirement for CUA immediately after the intron and for GGU immediately before the intron. Therefore, the resulting protein concatemer would have a glycine and leucine as a scar in the middle of the sequence upon circularization.

In some embodiments of pBAD-tdTEVDB, the Group I self-splicing intron requires the use of at least two co-factors. The co-factors are a divalent cation and/or a guanosine nucleoside. In some aspect, a guanosine is required because the intron uses a two-step mechanism that begins with the free nucleoside cleaving the phosphodiester linkage between the 5′-exon and the intron by prepending itself to the 5′-most nucleotides of the intron. In other aspect, a divalent cation is required to stabilize the tertiary fold of the catalytic core. In one aspect, the divalent cation is a magnesium cation. In some aspects, the divalent cation can be used in an amount from about 1 to about 30 mM, from about 5 to about 30 mM, from about 10 to about 30 mM, from about 15 to about 30 mM, from about 16 to about 30 mM, from about 17 to about 30 mM, from about 18 to about 30 mM, from about 19 to about 30 mM, from about 20 to about 30 mM, from about 15 to about 25 mM, from about 17 to about 25 mM, from about 18 to about 25 mM, from about 20 to about 25 mM, from about 21 to about 25 mM, from about 22 to about 25 mM, or from about 23 to about 25 mM to increase construct performance. In some aspects, the co-factors help the folding and stability of the intron.

In some embodiments, pBAD-tdTEVDB contains a mutation in at least one of a 36-base pair region covering a ribosome binding site (RBS), a ribosome biding site (RBS) spacer. an initiator codon, or a downstream box (DB) for purposes of increasing translational efficiency and product yield. In one aspect of pBAD-IdTEVDB, the initiator codon is an initiator methionine. In other aspect of pBAD-tdTEVDB, the mutation in the 36-base pair region is introduced using a modified error-prone PCR method which is which is described in Lee, S. O.; Fried, S. D. An Error-Prone PCR Method for Small Amplicons. Anal. Biochem. 2021, 628, 114266, the contents of which are herein incorporated by reference. The modified error-prone PCR method is based on a low-fidelity Mutazyme II DNA polymerase, iterating between dilution and reamplification, and touchdown PCR to suppress accumulation of incorrect product. In one aspect, the mutation is C-15A, G4C or C-15A/G4C. In some aspects. the mutation is double mutant C-15A/G4C in the initiation region.

In another embodiment, the present disclosure provides a modified construct in which a stop codon is inserted after the GFP coding sequence. The modified construct with a stop codon is designed to pBAD-tdTEVDB-STOP (See, FIG. 2C). In one aspect of pBAD-tdTEVDB-STOP, each ribosomal initiation event on circularized mRNA results in a single GFP protein being synthesized. The pBAD-tdTEVDB-STOP can be identical to pBAD-tdTEVDB except that a stop codon is placed at the end of GFP. More specifically, the pBAD-tdTEVDB-STOP can comprise in the following order: (i) a 3′ portion of a Group I self-splicing intron; (ii) a C-terminal portion of a super-folder GFP, wherein the C-terminal portion of the super-folder GFP comprises a 3′-exonic context sequence at its 5′ end; (iii) stop codon; (iv) a TEV protease cleavage site; (v) a ribosome binding site (RBS); (vi) a downstream box (DB) sequence; (vii) a N-terminal portion of a super-folder GFP, wherein the N-terminal portion of the super-folder GFP comprises a 5′-exonic sequence at its 3′ end; and (viii) a 5′ portion of the Group I self-splicing intron. In one aspect of the pBAD-tdTEVDB-STOP, the Group I self-splicing intron is a T4 bacteriophage thymidylate synthase (td) intron (herein after “T4 td intron”). In another aspect, the T4 td intron can be substituted with a Tetrahymena thermophila rRNA intron using routine techniques known in the art. In yet another aspect, the T4 td intron can be substituted with a Chlamydomonas reinhardii rRNA intron using routine techniques known in the art. In yet a further aspect, the T4 td intron can be substituted with a T4 bacteriophage sun Y intron, using routine techniques known in the art. In still further aspects, the T4 bacteriophage sun Y intron can be substituted with a Tetrahymena thermophila rRNA intron using routine techniques known in the art. In some aspects, the T4 td intron is circularly permutated at P6a for circulation.

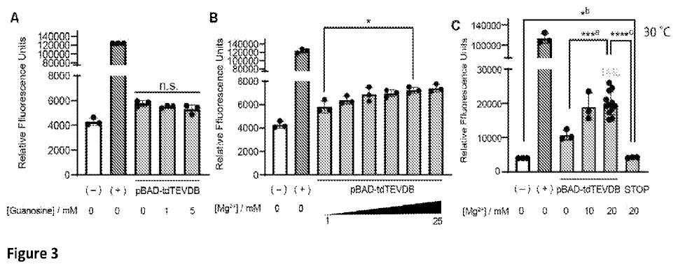

In one embodiment, the present disclosure results in increased efficiency of GFP fluorescence. In one aspect of pBAD-tdTEVDB, a fluorescence-based assay is used to probe the translational efficiency of circularized mRNAs. The translational efficiency can be calculated by the loop count. The loop count refers to the number of transits per initiation. In one aspect, the loop count is calculated using the following equation:

$$loop count = \frac{{F}_{loop} - {F}_{-}}{{F}_{stop} - {F}_{-}}$$

wherein F corresponds to average fluorescence, and “loop,” “−,” and “stop” corresponds to tdTEVDB, negative control, and tdTEVDB-STOP construct, respectively. The loop count (repeat number) from the loopable translators of the present disclosure can be very high (See, FIG. 3C), between about 50 to about 150.

### IV. Kits

In another embodiment, the present disclosure provides a kit comprising the genetic constructs described above in Sections II and III. Additionally, the kit can comprise at least two co-factors.

In some aspects of the kit, the co-factors are a divalent cation and/or a guanosine nucleoside. In some aspects, the divalent cation can be used in an amount from about 1 to about 30 mM, from about 5 to about 30 mM, from about 10 to about 30 mM, from about 15 to about 30 mM, from about 16 to about 30 mM, from about 17 to about 30 mM, from about 18 to about 30 mM, from about 19 to about 30 mM, from about 20 to about 30 mM, from about 15 to about 25 mM, from about 17 to about 25 mM, from about 18 to about 25 mM, from about 20 to about 25 mM, from about 21 to about 25 mM, from about 22 to about 25 mM. or from about 23 to about 25 mM.

### V. Method of Producing Biomaterials

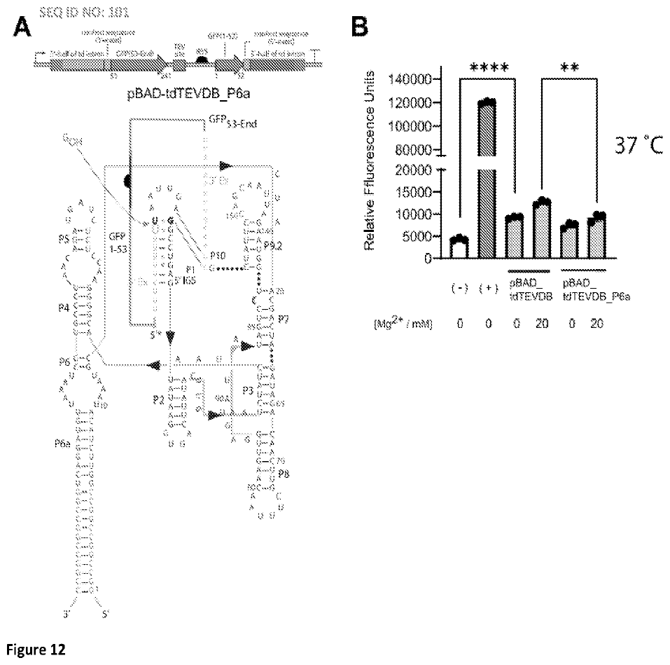

In yet another embodiment, the present disclosure provides a method of producing biomaterials. In certain embodiments, the present disclosure provides a method of producing a repetitive peptide chain. In another certain aspect, the present disclosure provides a method of producing a large repetitive peptide chain. The methods described herein comprise the step of transforming a host cell (such as a bacterial cell, a yeast cell, or a mammalian cell) with one or more of the genetic constructs described herein in Sections II and III, adding at least two co-factors, and culturing the host cell at a temperature of about 25° C. to about 37° C. to produce a biomaterial and/or repetitive peptide chain. In some aspects, high concentrations of certain co-factors (e.g., a divalent cation such as magnesium) and lower growth temperature are required for high levels of expression in order to help with the folding and stability of the intron. The benefit of the co-factors and temperature is supported by the higher RNA circularization levels (See, FIG. 12).

In some aspects of the method, at least two co-factors are added. In some aspects of the method, co-factors are a divalent cation and/or a guanosine nucleoside. In some aspects, the growth factor is a guanosine nucleoside. In other aspects, the co-factor is a divalent cation. In some aspects, the co-factors as a divalent cation and a guanosine nucleoside. In still further aspects, the divalent cation is a magnesium cation. In further aspects, the co-factors are a magnesium cation and a guanosine nucleoside.

In still another aspect of the method, the divalent cation, such as a magnesium cation, is added to the culture in an amount of from about 1 to about 30 mM, from about 5 to about 30 mM, from about 10 to about 30 mM, from about 15 to about 30 mM, from about 16 to about 30 mM, from about 17 to about 30 mM, from about 18 to about 30 mM, from about 19 to about 30 mM, from about 20 to about 30 mM, from about 15 to about 25 mM, from about 17 to about 25 mM, from about 18 to about 25 mM, from about 20 to about 25 mM, from about 21 to about 25 mM, from about 22 to about 25 mM, from about 23 to about 25 mM.

In another aspect of the method, the host cell is incubated at a lower growth temperature. Specifically, the host cell is incubated at a lower growth temperature, such as about from about 25° C. to about 37° C., about 25° C. to about 36° C., about 25° C. to about 35° C., about 25° C. to about 34° C., about 25° C. to about 33° C., about 25° C. to about 32° C., about 25° C. to about 31° C., about 25° C. to about 30° C., about 26° C. to about 30° C., about 27° C. to about 30° C., about 28° C. to about 30° C., about 29° C. to about 30° C. to produce the biomaterial. In another aspect, the host cell is incubated at a temperature of about 30° C. to produce the biomaterial.

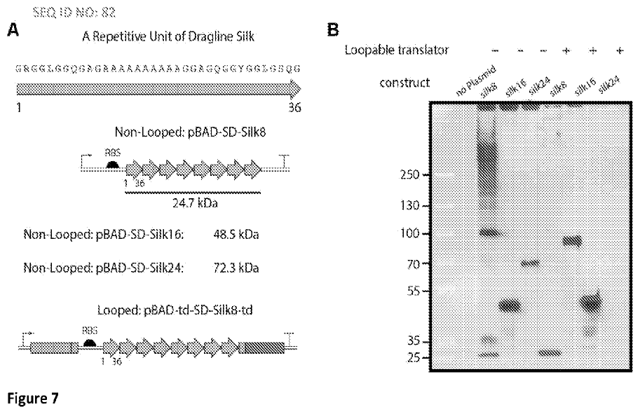

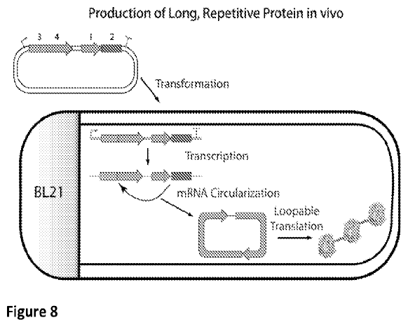

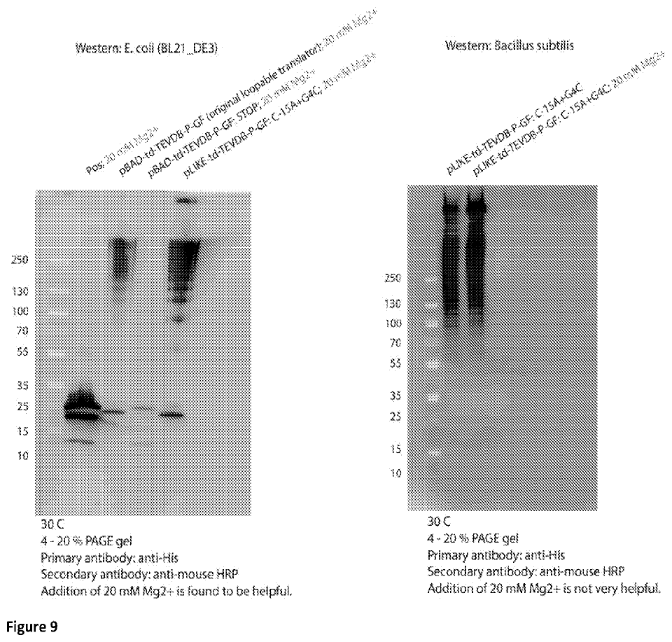

In some aspect of the method, biomaterial is made of protein produced by the loopable translator described herein in Sections II and III. In another aspect of the method, biomaterial comprises a repetitive peptide chain or protein produced by a loopable translator described herein in Sections II and III. In another aspect of the method, biomaterial comprised of high molecular-weight (250 kDa to 1 MDa) repetitive peptide chain or protein produced by a loopable translator described herein. In one aspect of the method, the biomaterial is dragline silk comprising spidroins. In some aspects, the sequences corresponding to the major ampullate spidroin protein 1 (MaSp1) are cloned into the loopable translator motif, and then high molecular weight spidroins are produced (See, FIG. 7A). In another aspect of the method, the biomaterial is a biofilm comprising curli proteins such as curli subunit A (CsgA). In yet another aspect of the method, the biomaterial is looped extracellular matrix (ECM) proteins such as fibronectin, laminin, collagen, reticulin, keratin, and elastin. In still yet another aspect of the method, the biomaterial is a squid ring teeth (SRT) proteins. In yet another aspect of the method, the biomaterial is globular cage-like protein nanomaterials. In yet a further aspect of the method, the biomaterial is any combinations thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example: Creation of a Loopable Translator Coupled to a Fluorescence Reporter

In one embodiment, a loopable translator is challenging to quantify, and indeed tandem repeats of green fluorescent protein (GFP) is not fluorescent (possibly because of efficient non-radiative energy transfer within the polymer). In one embodiment, super-folder GFP (sfGFP) was interrupted after its internal helix (at residue 52) with a T4 td intron (including 15 nucleotides of td exonic context flanking the intron). The exonic sequences were included to ensure proper interactions with the internal guide sequences (IGS) of the T4 td intron (circularly permuted at Poa for circularization; see, FIG. 2A-B). And then a ribosome binding site (RBS) was added before sfGFP's N-terminus, and a TEV protease cleavage site was added following sfGFP's C-terminus (See, FIG. 2A-B) and the sequence was cloned into an expression vector in which transcription was controlled by an arabinose promoter and a rrnB-T1 terminator. This plasmid (pBAD-td_{3′}-sfGFP_{53-end}-TEV-RBS-DB-sfGFP₁₋₅₂-td₅; note that the downstream box sequence (DB) refers to a sequence reported to enhance translational efficiency^{27,28}) was then co-transformed into E. coli BL21 (DE3) with pRK793 (expressing a soluble form of TEV protease (TEVP)).^{29,30} To abbreviate, the designed loopable translator construct is referred as pBAD-tdTEVDB. In this system, circularized mRNA is initially translated into a GFP concatemer; however, the activity of TEV protease (TEVP) liberates GFP monomers from the chain allowing for fluorescence to be monitored in vivo (See, FIG. 2B).

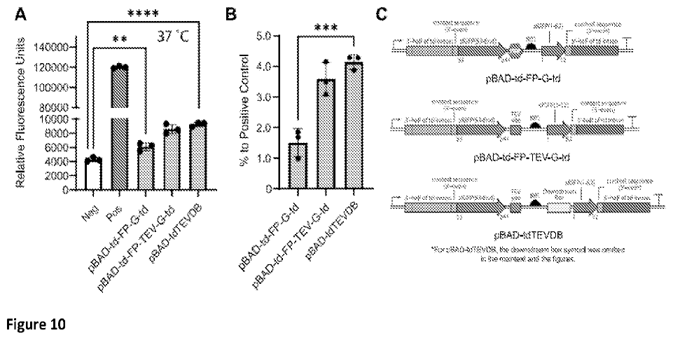

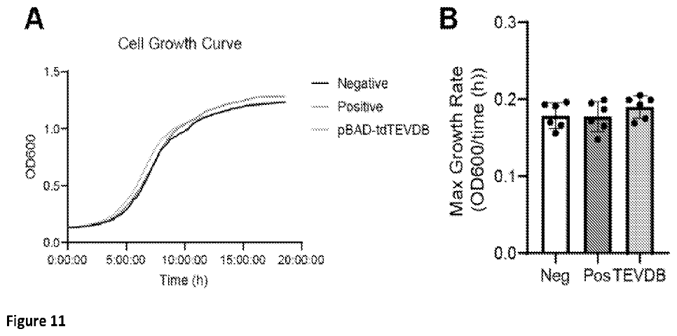

In one embodiment, pBAD-tdTEVDB was designed in a way that a non-fluorescent GFP fragment (residue 1-52) would be formed upon translation of a non-circularized RNA (translation was designed to terminate with a LAA stop codon that follows immediately after the end of 5′ exonic sequence (5′ Ex) (See, FIG. 2A-B). Thus. a failure of the mRNA molecule to circularize will result in truncated GFP, which is non-fluorescent and serves as a negative control (Sec. FIG. 2C). On the other hand, circularization (along with TEV protease activity) will result in full-length sfGFP with a 10 amino acid scar between residues 52 and 53, arising from the natural exonic sequences of td (shown in orange on FIG. 2A or as orange blocks in FIG. 2B-C). This GFP is still fluorescent, and it is used as a positive control (See, FIG. 2C). Preliminary fluorescence assays demonstrated that pBAD-tdTEVDB produces significant levels of fluorescence above baseline levels (See, FIG. 2D, FIG. 10; P<0.0001 by Student's t-test), confirming mRNA circularization occurs. Nevertheless, in one embodiment, fluorometric read-out in the present disclosure indicates that significantly less GFP (4.2%; see, FIG. 10) is produced compared to a positive control in which proteins are expressed via standard (vectorial) translation, suggesting that either (i) RNA circularization, (ii) ribosomal initiation, or (iii) TEV activity is limiting. To test for possible toxicities associated with loopable translation, the three constructs corresponding to negative control, positive control, and pBAD-tdTEVDB were transformed into BL21+pRK793 cells (with TEV protease). Three separate colonies were inoculated into 3×200 μL of LB media supplemented with ampicillin, tetracycline, 0.2% arabinose, IPTG (0.1 mM), and 20 mM Mg²⁺ in a clear-bottom 96-well plate (Costar) and expressed at 30° C. With the starting OD₆₀₀ of 0.05, the growth was measured in every 10 min over the course of 16 hours (See, FIG. 11). No significant difference was found in growth rates across these three strains.

Rational Improvement of Activity from Loopable Translator

In one embodiment, this example is to improve the activity of pBAD-tdTEVDB by considering a number of factors that could influence the three considerations above. Group I self-splicing introns require two cofactors: magnesium cations and a guanosine nucleoside.^{31,32} In one embodiment, GFP expression from pBAD-tdTEVDB was not strongly dependent on guanosine (see, FIG. 3A): however, fluorescence could be markedly improved by 147% (from 5.09% to 7.46% of the positive control) by increasing the concentration of MgCl₂ in the growth media up to 20 mM (see, FIG. 3B). These findings are broadly consistent with the facts that intracellular total guanosine ((p) (p) (p) Gon) concentration is quite high in E. coli (5 mM, higher than the intron's K_{M}³⁵); however, free Mg²⁺ in the cytosol is not at high concentrations (˜1 mM).

In one embodiment, an enhancement in GFP expression was observed when cells were incubated at a lower growth temperature (30° C.), with the fluorescence levels approaching 16% of positive control (See, FIG. 3C). In another embodiment, two potential reasons are speculated why this could occur: (i) TEV protease (TEVP) is an aggregation prone protein, and reduced temperatures could improve its solubility in the E. coli cytoplasm; and (ii) lower temperatures could shift equilibria to favor certain catalytic elements on the intron, some of which consist of only two basepairs (e.g., P10; see, cf. FIG. 2A).³⁶

As a control, a fluorescence assay was also performed with a modified construct in which a stop codon is inserted after the GFP coding sequence (pBAD-tdTEVDB-STOP, see FIG. 2C), such that each ribosomal initiation event on circularized mRNA would only result in a single GFP protein being synthesized (non-circularized mRNA would still result in truncated GFP). Fluorescence levels decreased markedly, down to levels only slightly higher than the baseline level associated with the negative control (See, FIG. 3C). This result suggests that the fluorescence signal achieved by pBAD-tdTEVDB arises from ribosomes transiting circular mRNAs many times without stopping. Using the slightly higher (but still statistically significant) fluorescence signal of pBAD-tdTEVDB-STOP relative to the negative control (1.04-fold, P-value=0.04 by Student's t-test), the loop count (number of transits per initiation) is estimated to be ˜90±55 using:

$$loop count = \frac{{F}_{loop} - {F}_{-}}{{F}_{stop} - {F}_{-}}$$

wherein F corresponds to average fluorescence, and “loop,” “−,” and “stop” correspond to the tdTEVDB, negative control, and tdTEVDB-STOP constructs, respectively.
   Visualization of Concatemer Formation and Verifying mRNA Circularization

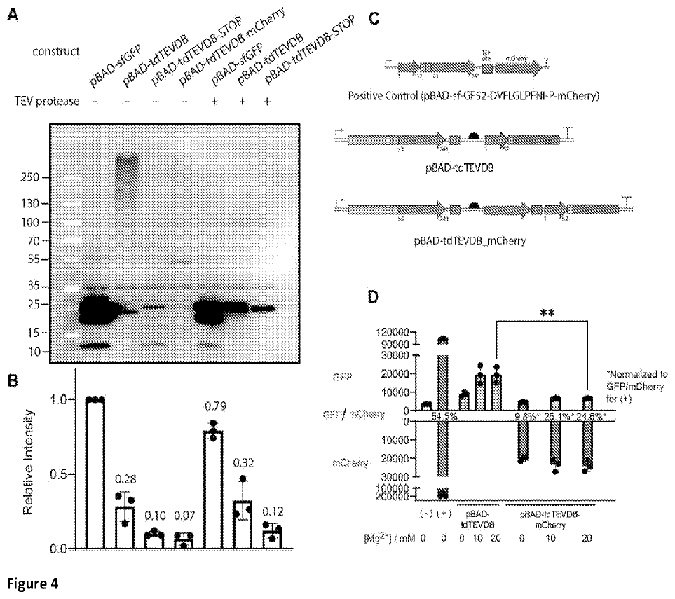

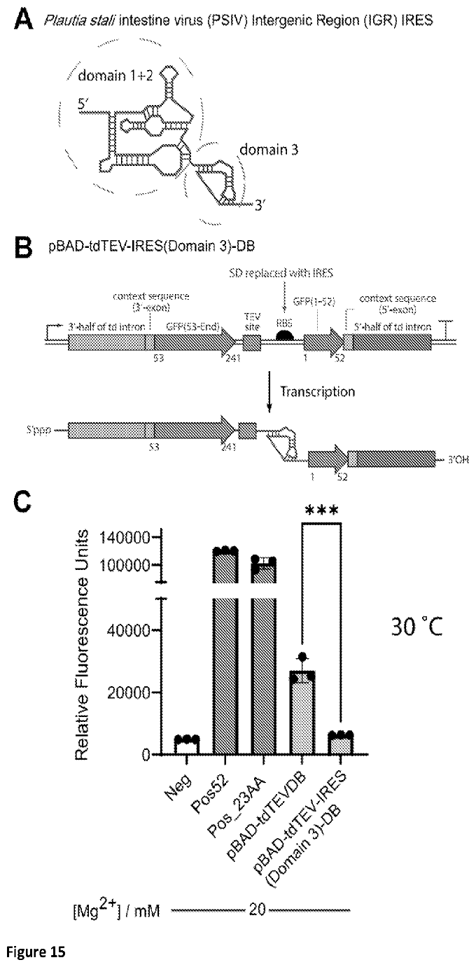

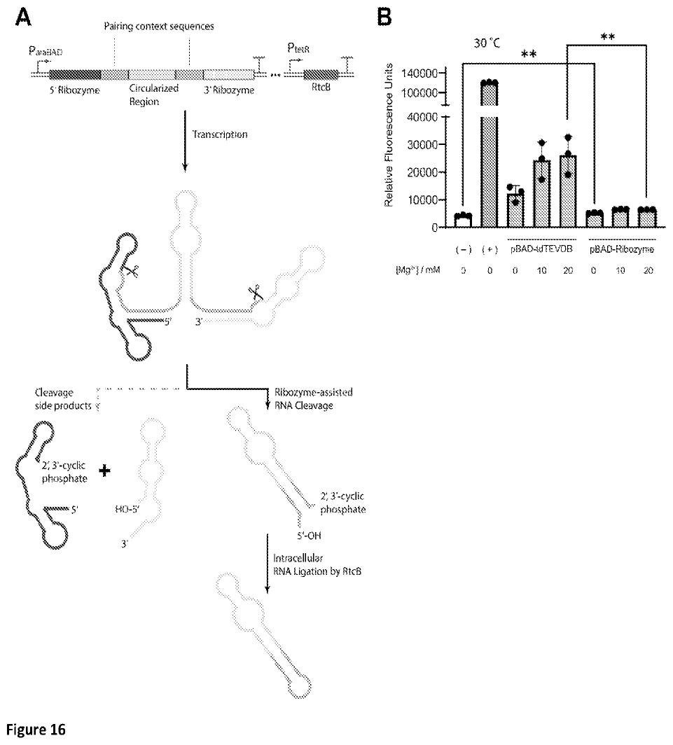

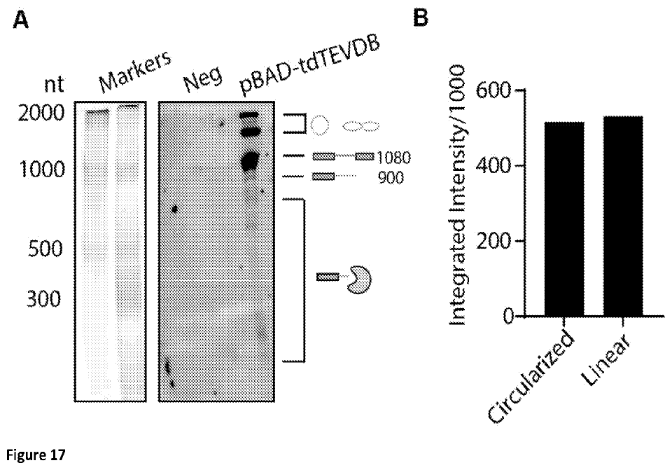

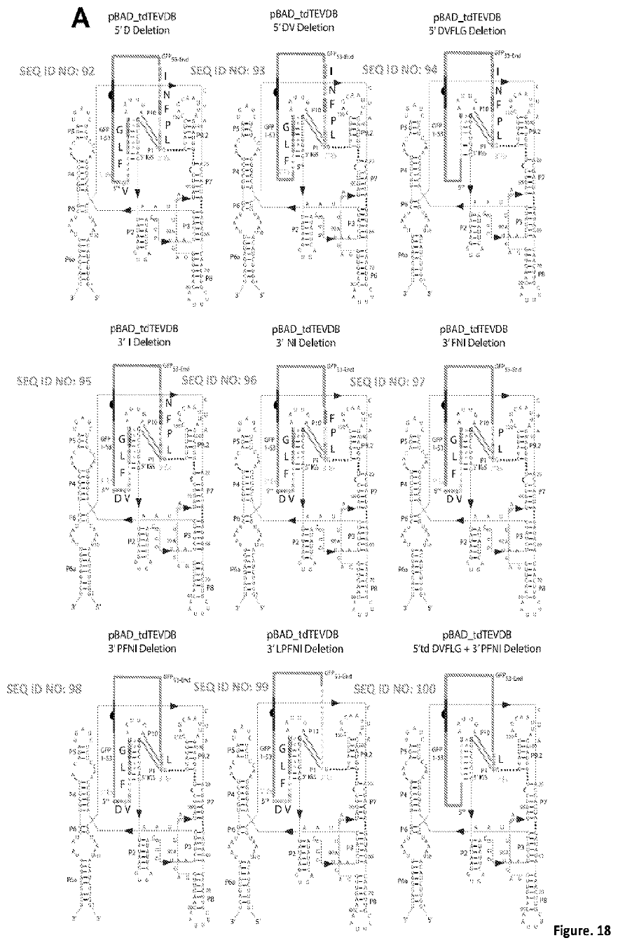

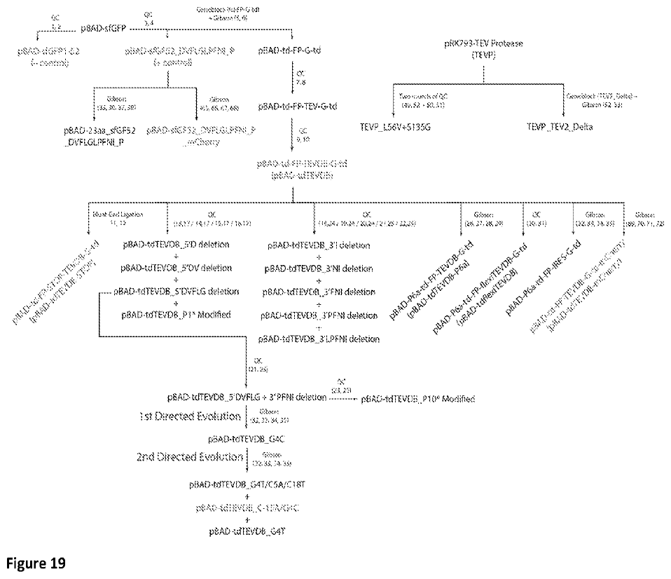

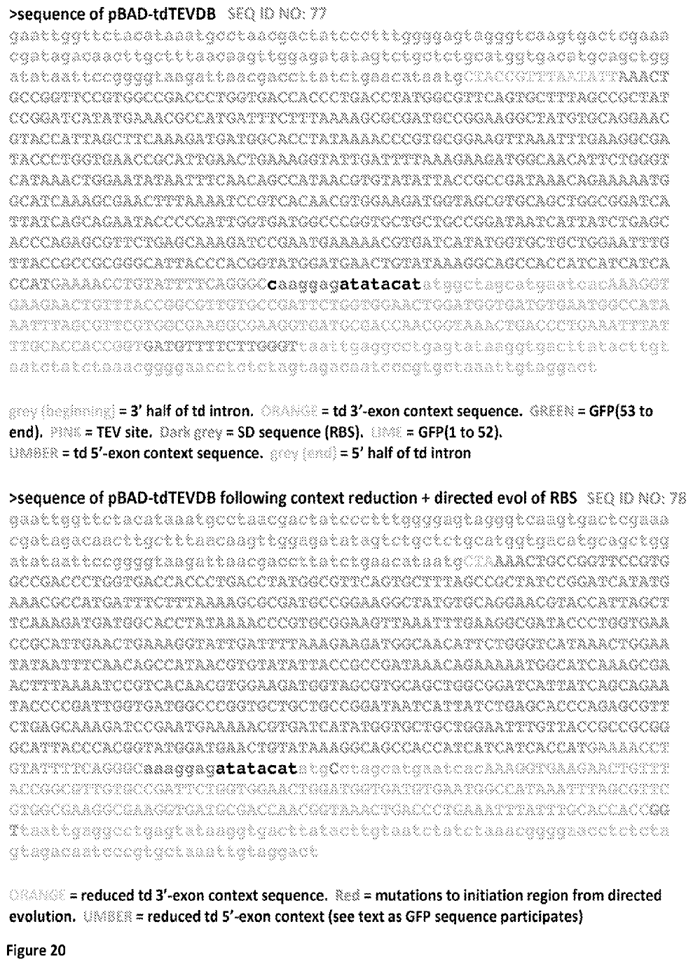

Because polymeric GFP (polyGFP) is not fluorescent.²³ the fluorescence assays require the activity of TEV protease (TEVP) to generate a readable signal. In contrast, polyGFP can be visualized directly as a protein of high molecular weight via Western blot. In one embodiment, in the absence of the plasmid that expresses TEV protease (TEVP), pBAD-tdTEVDB generates a set of protein products with high molecular weights (>250 kDa) that are specific for an anti-His antibody (the GFP coding sequence contains a His-tag, see FIG. 4A, FIG. 15). In contrast, in the presence of TEV protease (TEVP), pBAD-tdTEVDB generates a single band at the expected molecular weight for monomeric GFP (25 kDa), identical to a positive control in which GFP is expressed by linear translation (See, FIG. 4A). Moreover, the introduction of a stop codon within the mRNA loop completely abrogates the high-molecular weight features, as expected, and also significantly reduces protein expression level-showing that polyGFP synthesis is dependent on a ribosome iterating numerous times on circular mRNA. Densitometry analysis conducted on blots of biological triplicates (see, FIG. 4B) showed that relative to linear translation, overall protein expression from the loopable translator was down ˜3.6-fold, and protein expression from pBAD-tdTEVDB-STOP was down ˜10-fold, consistent with the data in fluorescence assays (See, FIG. 3C).

To provide independent verification that mRNA is circularized by the split-intron architecture, designed a dual-fluorescence ratiometric construct was designed (See, FIG. 4C). In one embodiment, the construct, called pBAD-tdTEVDB-mCherry, places a full-length mCherry open reading frame between the RBS and the N-terminal fragment of sfGFP. In this construct, mCherry is expressed independently of circularization while GFP, as before, requires mRNA circularization to be fully synthesized. From this construct, GFP signal was 4-fold less than that of mCherry (after correcting for background and intrinsic difference in sfGFP's and mCherry's fluorescence intensity, see FIG. 4D), suggesting that 25% of the tdTEVDB-mCherry mRNA is circularized in the steady state. In further embodiments, overall GFP fluorescence signal in this larger 1563-nt loop was significantly reduced (3-fold) relative to the smaller 816-nt loop created by pBAD-tdTEVDB, which is consistent with the Western blot showing 4-fold reduced polyGFP from pBAD-tdTEVDB-mCherry relative to pBAD-tdTEVDB (See, FIG. 4A-B). In one embodiment, forming the larger loop has a higher entropy cost to bring the two portions of the split-intron together and assemble the active ribozyme. If this difference in GFP signal can be ascribed solely to the fraction of mRNA molecules that are circularized, these data suggest that as much as ˜75% of the tdTEVDB mRNA is circularized in the steady state. Although there could be other factors involved in GFP's translational efficiency from these two circular RNAs, Northern blot analysis of the RNA from cells harbouring pBAD-tdTEVDB found that 50% of the GFP-containing RNA was circularized (See, FIG. 12). In one embodiment, evidence from Western blots, Northern blots, and the dual-fluorescence assay support the view that the split-intron can achieve a steady-state fractional circularization level of 50-70% of 800-nt regions. In one embodiment, this level suitable for the present disclosure's purposes, though it is noted that fractional circularization could be potentially optimized with further directed evolution of the intron.

### Identifying the Minimal Context Requirements for Circularization

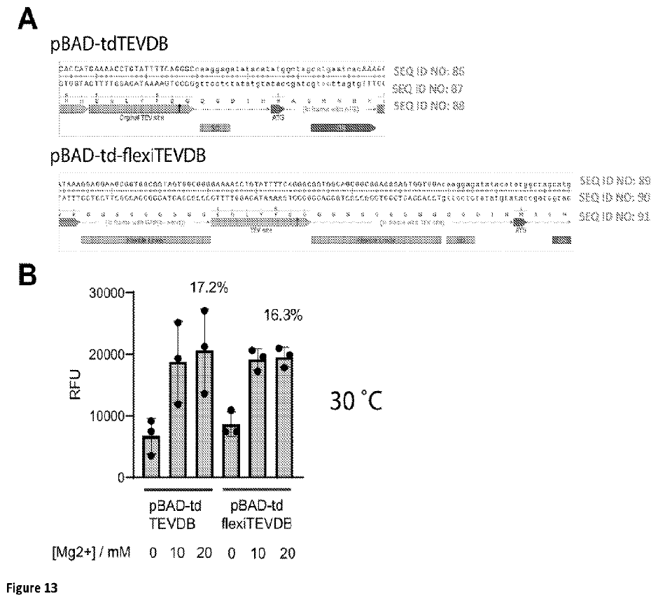

In one embodiment, the design of pBAD-tdTEVDB would incorporate a 10 amino acid scar at the junction site where looping occurs. In one embodiment, the minimal “exon context” requirements for intron activity were performed by “walking back” the 15 nucleotides of native exon context on each flank. Classic experiments on self-splicing introns demonstrated the essentiality of the final U·G wobble pair at the 5′ splice-site and the formation of the P1 stem between the 5′-exon and the beginning of the intron sequence (5′ IGS).²⁵ The td's 5′-exon context (GAU/GUU/UUC/UUG/GGU, encoding DVFLG) one codon at a time (See, FIG. 5A, C & FIG. 13) was successively deleted and remarkably, all 5 codons of td's 5′-exon context could be removed, resulting in a slight improvement in fluorescence activity. Inspection of the RNA molecule created following this deletion revealed that coincidentally sfGFP's residues 50-52 are Thr-Thr-Gly, which are encoded by ACC/ACC/GGU, a sequence that conserves the three critical nucleotides (GGU) immediately before the intron (forming two strong base pairs, then a wobble base pair, See, FIG. 5B). Moreover, like the wild-type intron, this sequence creates four total Watson-Crick basepairs along P1. In one embodiment, IGS nucleotides (U12 and A14, counting from the first nucleotide of the intron) to basepair with the native sfGFP nucleotides (P1* in red in FIG. 5B) were mutated, however, the mutation reduced fluorescence down to baseline levels (See, FIG. 5C), suggesting that it abrogates circularization activity. These observations imply that the presence of several (possibly two) non-Watson-Crick basepairs in P1 is necessary for activity. In one embodiment, the minimal requirements for proper selection of the 5′ exon fragment by P1 are: (i) it must end in GGU; and (ii) the preceding four nucleotides must make two Watson-Crick (but not more) basepairs with the intron's 5′-IGS.

The td's 3′-exon context region one codon at a time (See, FIG. 5A, D; FIG. 13) was abrogated and it was confirmed that only the initial three nucleotides (CUA; the ones closest to the exon and marked in magenta) are required for activity. A deletion of the first 3 nucleotides of the 3′-exon (CUA) abrogated all circularization activity (See, FIG. 5D), confirming the importance of these nucleotides for splice site selection and therefore circularization.

Putting these observations together, the present disclosure is to identify the minimal exon context requirements for intron activity, so that future use of the loopable translator system would impose minimal scar sequences at the junction site (See, FIG. 5E). In one embodiment, complete removal of the 5′ td-exon context and removal of the 3′ td-exon context up to the first three nucleotides (CUA) were tolerated by the permuted td intron, and in fact provided a small (20%) activity increase with respect to the wild-type (Sec. FIG. 5E). In another embodiment, to determine if intron activity could be enhanced by strengthening P10, the sequence of the initial three nucleotides from CUA to CUC, which would form a third Watson-Crick basepair with the P1 loop (P10* in blue in FIG. 5B, E), was modified. The resulting construct generated signal that was slightly lower than the construct with only CUA, suggesting that two basepairs of complementarity is indeed the optimal strength for this critical interaction. In one embodiment, combining the requirement for CUA immediately after the intron, and the requirement of GGU immediately before the intron, data in the present disclosure imply that mRNA circularization can be accomplished with a scar as small as two amino acids at the junction region.

Improving Initiation on Circular mRNA by Directed Evolution

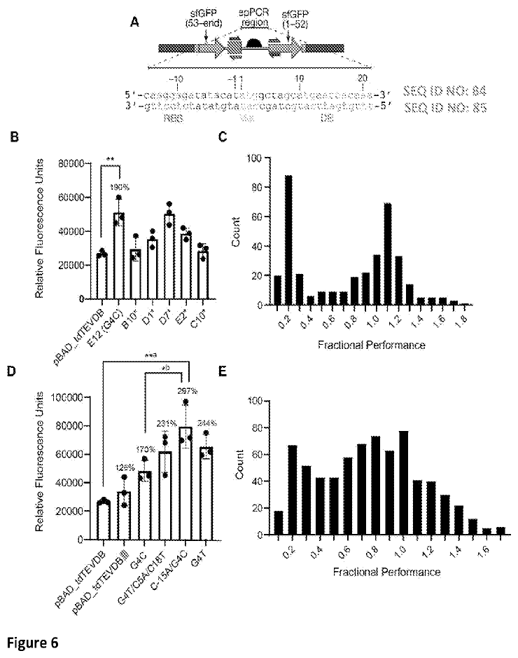

In this disclosure, it was examined whether directed evolution on the initiation region (See, FIG. 6A) would enable the creation of a modified initiation sequence better suited for circular mRNA.

Specifically, several mutations were introduced into a short 36-bp region that covers the canonical RBS, its spacer, the initiator codon, and the downstream box. Conventional error-prone PCR methods were ill suited for achieving the desired mutation density in this small amplicon (1-2 mutations per 36 bp) while also maintaining low levels of bias;⁴⁷⁻⁵⁰ The modified error-prone PCR method based on a low-fidelity Mutazyme II DNA polymerase, iterating between dilution and reamplification, and touchdown PCR to suppress accumulation of incorrect products were used. The details of this modified method are herein incorporated by reference 51.

This dilution/reamplification error-prone PCR method was used to introduce mutations into a 36-basepair region, which was then cloned back into the pBAD-tdTEVDB vector linearized with the reverse complement primers (red primers, in FIG. 6A), via In-Fusion cloning (see, the Materials and Methods section). The library DNA was isolated and re-transformed into BL21 (DE3) cells harbouring the pRK793 plasmid, and hundreds of colonies were selected from agar plates at random for inoculation into 96-well plates to screen for beneficial mutations by fluorescence. In the first screen, fluorescence from 372 constructs was assayed and a bimodal distribution (See, FIG. 6C) with many clones having a neutral phenotype (fractional performance close to 1) or with very low activity (fractional performance close to 0.2) was shown. A small number of variants had improved levels of translational efficiency, up to 1.9-fold, including a variant E12 (which contained the mutation G4C, immediately following initiator methionine; See, FIG. 6B). Because other high performers by this screen contained mixtures of sequences upon performing sequence analysis, G4C was used as a starting point for a second round of diversification and screening. In this present disclosure the pBAD-tdTEVDB^{G4C} plasmid was used as a template for error prone PCR to incorporate further mutations in the initiation region and cloned back into the pBAD-tdTEVDB backbone to perform a second screen. This time, 720 constructs were assayed, and that mutants that improved translational efficiency 1.8-fold again (See, FIG. 6E) were discovered. The best performing mutant, C-15A/G4C contains an additional mutation that apparently “extends” its RBS sequence to now include a run of 8 purines; other successful clones contained other mutants at position 4. Overall, the double mutant C-15A/G4C in the initiation region is ca. 3-fold more active at producing GFP relative to the starting construct (See, FIG. 6D). In one embodiment, these experiments appear to support ribosomal initiation rates hampered translation on circular mRNA, though specific alterations to the initiation region can mitigate this limitation. Finally, if these improvements in translational initiation with lowered temperature and increased Mg²⁺ concentrations are combined, the result is a ˜14-fold improvement in translational efficiency relative to the original loopable translator (See, FIG. 6D and FIG. 2D, ca. 75000 and 5260 relative fluorescence units, respectively, following background subtraction). Overall, protein expression from these optimized circular mRNAs is within a factor of 1.5 relative to the expression levels from standard linear mRNA.

### Discussion

Loopable translation has several traits that could make it an attractive option for the preparation of highly repetitive proteins. Because it does not require the creation of repetitive DNA sequences, the resulting genetic constructs would be expected to be more stable and less susceptible to homologous recombination. Moreover, the absence of repetitive sequences renders these plasmids amenable to many more molecular biology and synthetic biology manipulations-such as PCR, Gibson assembly, and recombineering-which are challenging (or impossible) to conduct with highly repetitive DNA sequences. These features should allow for greater interoperability; that is, it is facile for any researcher to “swap out” the GFP sequence in pBAD-tdTEVDB with an arbitrary sequence via a single Gibson assembly. All of these features additionally make this system much more amenable to creating combinatorial libraries and performing directed evolution. Due to the universal nature of the autocatalytic intron splicing reaction (requiring only magnesium and guanosine as cofactors), circularization should be functional in a range of microbial hosts. Finally, the repeat number (loop count) from such a system could be very high—as the data with the pBAD-tdTEVDB-STOP construct (FIG. 3C) would suggest—possibly only limited by the presumably rare occurrence of spontaneous frame-shifting⁵² or possibly non-specific peptide release from the ribosome.

### Materials and Methods Used for Example

No unexpected or unusually high safety hazards were encountered.

Cloning pBAD-tdTEVDB Plasmid Construct and Controls

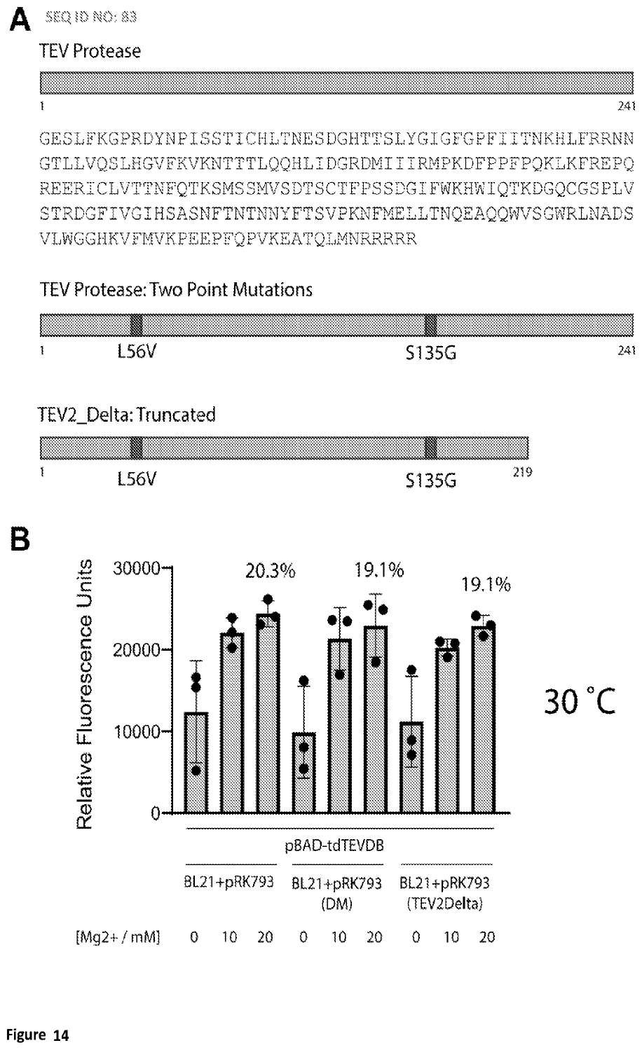

To create the negative control plasmid (pBAD-sfGFP1-52), a plasmid encoding the full-length superfolder GFP (pBAD-sfGFP) was used as a template for PCR and amplified with primers Delete53-f and Delete53-r (See, Sequence Listing; FIG. 14) using the Q5 DNA polymerase (NEB) according to the manufacturer's protocol. The construct was designed in a way that the truncated GFP is under the control of the same arabinose-inducible promoter and terminator. The PCR product was assessed by 0.8% agarose (0.5×TBE) gel electrophoresis for the correct molecular weight, DpnI (NEB) digested (10 U were added to the PCR reaction, and incubated at 37° C. for 30 min, then 80° C. for 20 min to inactivate), column purified using the DNA clean-and-concentrate kit (Zymo) according to the manufacturer's protocol and quantified using a Nanodrop OneC (Thermo). The DNA was ligated using the QuickChange strategy by directly transforming it into chemically competent 10-beta cells (NEB) without in vitro ligation. PCR products that are described as being ligated by QuickChange mean that the primers were designed to encode ˜15 nucleotides of homology at the 5′ and 3′ termini, enabling ligation in vivo by 10-beta cells' endogenous recombinases (See, Sequence Listing for details). Next, 1 μL of purified DNA was combined with 25 μL of competent cells, incubated on ice for 25 min, subject to heat pulse at 42° C. in a water bath for 40 s, and then returned to ice for 2 min. Next, cells were recovered by inoculation into 1 mL of SOC media and incubated at 37° C. for 1 h with agitation (700 rpm in a thermomixer), and then 100 μL of the transformant was spread out on selective plates consisting of LB Agar supplemented with 15 μg/mL tetracycline using coli rollers. After ˜16 h incubation at 37° C., colonies were selected and inoculated into 5 mL LB supplemented with 15 μg/mL tetracycline and incubated overnight (˜16 h) at 37° C. with agitation (220 rpm). Cells from the saturated overnight cultures were collected by centrifugation (3200 g for 15 min at 4° C.), and plasmid DNA was isolated using the ZR plasmid miniprep kit (Zymo) according to the manufacturer's protocol. In this (and in all further molecular cloning manipulations), plasmid DNA would be typically isolated from 4-6 colonies and subject to sequencing analysis by Sanger sequencing (GeneWiz, following GeneWiz's specifications). A single clone with the correct DNA sequence would then be used for further steps.

The positive control was created through a QuickChange approach to introduce the two td-exon context sequences (encoding the decapeptide DVFLGLPFNI) at the junction between residues 52 and 53. PCR was conducted on pBAD-sfGFP as template and using the primers TD_Context_Insert-f and TD_Context_Insert-r (See, Sequence Listing; FIG. 14). The PCR product was purified and transformed as described above. This construct creates a replica of the GFP molecule that would be created by the loopable translator.

A geneBlock for the prototype Loopable Translator (td-FP-G-td) was ordered from IDT and cloned after the araBAD promoter in pBAD through Gibson Assembly. A linearized pBAD vector was generated by PCR using pBAD-sfGFP as template, and oligos clone-pBADGFP-f and clone-pBADGFP-r as primers. The vector fragment was assessed by 0.8% agarose (0.5×TBE) gel electrophoresis for the correct molecular weight, DpnI (NEB) digested (10 U were added to the PCR reaction, and incubated at 37° C. for 30 min, then 80° C. for 20 min to inactivate), and column purified (Zymo). The prototype geneBlock was designed to have the permuted sfGFP (residues 53-241, then 1-52) flanked by the permuted td intron of T4 bacteriophage (P6a-P9.2, P1-P6a). The td-FP-G-td geneblock has a His tag at the end of GFP residue 241, a stop codon at the end of the His tag, and ribosome binding site (RBS) in front of GFP residue 1. The geneblock and the linearized vector were ligated using HiFi 2× Gibson Master Mix (NEB) according to manufacturer's protocol and transformed into chemically competent 10 beta cells as described above.

Because polyGFP is not fluorescent,^{23,24} this system was modified in a few more ways. A TEV cleavage site was installed after the His-tag and the stop codon was removed using PCR with primers TEV-f and TEV-r via QuikChange. The downstream box sequence^{27,28} was also installed after the start codon using PCR with primers InsertDB-F and InsertDB-R via QuikChange (See, Sequence Listing; FIG. 14). Following this sequence of alterations, the resulting construct corresponds to the pBAD-tdTEVDB drawn in FIG. 2C and interrogated in FIG. 3. The full nucleotide sequence of this construct is given in FIG. 15.

To create pBAD-tdTEVDB-STOP, the TAA stop codon was reintroduced between GFP and the TEV site by PCR with primers TEV_DB_STOP_F and TEV_DB_STOP_R via blunt-end ligation (See, Sequence Listing; FIG. 14).

### Fluorescence Assays

First, chemically competent BL21 (DE3) (NEB) cells were transformed with pRK793 (encoding TEV protease)³⁰—a gift from the laboratory of Doug Barrick (JHU Biophysics department). 1 μL of pRK793 plasmid DNA and 25 μL of competent BL21 (DE3) cells were mixed together in a microfuge tube by aspiration, incubated on ice for 25 min, subject to heat pulse at 42° C. in a water bath for 40 s, and then returned to ice for 2 min. Next, cells were recovered by inoculation into 1 mL of SOC media and incubated at 37° C. for 1 h with agitation (700 rpm in a thermomixer), and then 100 μL of the transformant was spread out on selective plates consisting of LB Agar supplemented with 100 μg/mL ampicillin using coli rollers. After ˜16 h incubation at 37° C., one colony was inoculated into 5 mL of LB supplemented with 100 μg/mL ampicillin and incubated overnight (˜16 h) at 37° C. with agitation (220 rpm). The overnight culture was inoculated into 1 L of LB with 100 μg/mL ampicillin in a 2-L baffled sterile flask to a starting OD₆₀₀ of 0.02-0.04 to make homebrew BL21 (DE3)+pRK793 competent cells. The BL21 (DE3)+pRK793 day culture was incubated at 37° C. with agitation (220 rpm) until the OD reached 0.4-0.6. When the desired OD was achieved, the culture was transferred into 500 mL centrifuge bottles and incubated on ice for 20 min (after this step, the culture was either handled on ice or in a cold room). During the incubation, three buffers (200 mL of 100 mM MgCl₂, 200 mL of 100 mM CaCl₂), and 60 mL of 85 mM CaCl₂), 15% glycerol) were prepared and pre-chilled on ice. The ice-incubated culture was centrifuged at 3000 g for 15 min at 4° C. The supernatant was decanted off and cell pellets in each 500 ml bottle were resuspended in 100 mL of cold 100 mM MgCl₂. The resuspension was centrifuged at 2000 g for 15 min at 4° C. The supernatant was decanted off and cell pellets in each 500 ml bottle were resuspended in 100 mL of cold 100 mM CaCl₂). The resuspension was incubated on ice for 20-40 min and centrifuged at 2000 g for 15 min at 4° C. The supernatant was decanted off and cell pellets in each 500 ml bottle were resuspended in 25 mL of cold 85 mM CaCl₂). 15% glycerol. transferred to a 50 mL falcon tube and spun at 1500 g for 15 min at 4° C. The supernatant was decanted off and cell pellets in the 50 mL tube were resuspended in 4 mL of cold 85 mM CaCl2.15% glycerol. The 4 mL of competent BL21 (DE3)+pRK793 cells were aliquoted into 40 microfuge tubes, containing 105 μL apiece, and were flash frozen in liquid nitrogen and stored at 80° C. until further use.

To begin a fluorescence assay, an aliquot of BL21 (DE3)+pRK793 was thawed on ice. 1 μL of pBAD plasmid DNA and 25 μL of competent BL21 (DE3)+pRK793 cells were mixed together in a microfuge tube by aspiration, incubated on ice for 25 min, subject to heat pulse at 42° C. in a water bath for 40 s, and then returned to ice for 2 min. Next, cells were recovered by inoculation into 1 mL of SOC media and incubated at 37° C. for 1 h with agitation (700 rpm in a thermomixer), and then 40 μL of the transformant was spread out on selective plates consisting of LB Agar supplemented with 100 μg/mL ampicillin and 15 μg/mL tetracycline using coli rollers. After ˜16 h incubation at 37° C., (typically) three separate colonies were inoculated into 3×200 μL LB supplemented with 100 μg/mL ampicillin and 15 μg/mL tetracycline in a clear-bottom 96-well plate (Costar). After inoculation, the plate was sealed with parafilm and incubated overnight (˜16 h) at 37° C. with agitation (220 rpm), and these created the biological triplicates used for fluorescence measurements. For repeat measurements, separate colonies would be selected from the same agar plate, which were stored at 4° C. and would be reused for at most 1 week.

The final OD₆₀₀ were measured for the overnight cultures using a plate reader instrument (SpectraMax iD3 from Molecular Devices) and were used to subculture down to a starting OD₆₀₀ of 0.05 in 200 μL of induction media (LB supplemented with 15 μg/mL tetracycline, 100 μg/mL ampicillin, 0.1 mM IPTG, and 0.2% arabinose) in a clear-bottom 96-well plate. 96-well plates containing biological triplicates for each of the conditions considered would be loaded into an iD3 microplate reader preincubated at 37° C. The plate reader recorded both growth (OD₆₀₀ by absorbance) as well as GFP fluorescence (excitation at 488 nm, emission at 535 nm, PMT gain at 500 volts with integration time of 20 ms from 5 mm from the plate) every 10 min over 16 h. In between measurements, the plate reader agitated the plate with orbital mixing (High; 577 rpm).

In later experiments, plate reader measurements would be conducted at 30° C. (instead of 37° C.). Additionally, induction media was additionally supplemented with various concentrations of guanosine (Sigma) or MgCl₂.

To analyze the data, the absorbance graphs of the cultures were first examined to see if any data points needed to be removed due to absence of growth. Based on early experiments, it was found that the fluorescence time point after 50,000 sec of growth was representative of maximal expression levels before cell death, and hence this timepoint was used throughout this study. Fluorescence values at 50,000 sec were compiled in Graphpad Prism 9, which was used to generate the bar charts shown in FIGS. 2, 3, 4, 5, 6, and 10. Statistical analysis was conducted using Student's t-test (assuming normally distributed populations with equal variances), as implemented in Prism 9.

### Protein Expression and Western Blot

1 μL of each pBAD plasmid DNA was mixed together with either 25 μL of competent BL21 (DE3) cells or 25 μL of competent BL21 (DE3)+pRK793 cells, respectively, in a microfuge tube by aspiration, incubated on ice for 25 min, subject to heat pulse at 42° C. in a water bath for 40 s, and then returned to ice for 2 min. Next, the cells were recovered by inoculation into 1 mL of SOC media, incubated at 37° C. for 1 h with agitation (700 rpm in a thermomixer), and 40 μL of the transformant was spread out on corresponding selective plate using coli rollers; BL21 (DE3) transformants were spread on selective plate consisting of LB Agar supplemented with 15 μg/mL tetracycline only, and BL21 (DE3)+pRK793 transformants were spread on selective plate consisting of LB Agar supplemented with 100 μg/mL ampicillin and 15 μg/mL tetracycline. One colony from each plate was inoculated into 5 mL LB supplemented with the corresponding antibiotics in a 14-mL sterile round bottom tube (ThermoFisher) and grown overnight (˜16 h). These overnight cultures were sub-cultured down to a starting OD₆₀₀ of 0.05 in 50 mL of LB media supplemented with corresponding antibiotics, inducers (0.2% arabinose and 0.1 mM IPTG), and 20 mM Mg²⁺ in sterile 250 mL Erlenmeyer flasks. The day cultures were grown for ˜4 h at 30° C. with agitation (220 rpm) to final ODs of 1.0 to 1.2, aliquoted into 1.5 mL microfuge tubes by 1 mL, spun down at 3000 g for 15 min at 4° C., and stored at −20° C. until future use.

For lysis, the cell pellets were thawed on ice for 15 min and resuspended in 1 mL of 1×PBS. 30 μL of each resuspension was transferred to a microfuge tube, mixed with 7.5 μL of 5× Tris-Glycine-SDS loading buffer via vortexing, heated in a 90° C. water bath for 5 min, and incubated on ice for 2 min. 30 μL of each lysate was loaded onto pre-cast Novex WedgeWell 8-16% Tris-glycine Mini Protein gels (ThermoFisher Scientific) with 3 μL of Pre-stained PAGE ruler as the ladder (ThermoFisher Scientific; #26619) and were separated by electrophoresis at 140 V for approximately 1 h, using 1× Tris-glycine-SDS electrophoresis running buffer (BioRad). The resulting gel was incubated for approximately 2-3 min in 0.8× Tris-glycine buffer (BioRad), 20% (v/v) methanol. The gel was trimmed to remove the wells and foot, and electroblotting was performed using iBlot 2 Gel Transfer Device (ThermoFisher) and its matching transfer packet (Invitrogen; iBlot 2 Transfer Stacks, PVDF, regular size), according to the manufacturer's protocol (7 min; 20 V). After electroblotting, the PVDF membrane was incubated in 15 mL of 5% (w/v) nonfat milk (Nestle-Carnation. Instant Nonfat Dry Milk)-TBST solution for approximately 1 h with rocking to block the membrane. 5% nonfat milk-TBST solution was made by combining 1×TBS (Quality Biological; pH 7.4) with evaporated milk and Tween 20 to a final concentration of 0.1% (v/v). The blocked membrane was incubated ˜16 h in 8 mL of diluted primary anti-His antibody (mouse; Invitrogen) solution at 4° C. with rocking; the diluted primary antibody was made by diluting the antibody by 1:1000 in 5% (w/v) nonfat milk-TBST. The membrane was rinsed in 1×TBST 3 times for 10 min each, incubated in 8 mL of diluted secondary anti-mouse-HRP antibody (goat; Invitrogen) solution at room temperature for 40 min with rocking; the diluted secondary antibody was made by diluting the antibody 1:10,000 in 5% (w/v) nonfat milk-TBST. The incubated membrane was rinsed in 1×TBST 3 times for 10 min each, incubated for 1 min in 800 μL of chemiluminescence reagents (Signal West Femto Maximum Sensitive Substrate; ThermoFisher Scientific) that were mixed in a 1:1 ratio, and then images were acquired using a ChemiDoc Touch Imaging System (BioRad).

### Making Constructs for a Dual-Fluorescence Ratiometry Assay

A pRK5 plasmid containing a gene that encodes mCherry was obtained as a gift from the laboratory of TackJip Ha (JHU Biophysics department). Using this plasmid as a template, two new constructs were made by PCR via Gibson assembly: a new positive control with mCherry (pBAD-sfGFP52-DVFLGLPFNI-P-mCherry) and pBAD-tdTEVDB with mCherry (pBAD-td-FP-TEVDB-mCherry-TEV-G-td; denoted as pBAD-tdTEVDB-mCherry) (See, FIG. 4C). For the new positive control, a linearized vector was made by PCR using the original positive control (pBAD-sf-GF_DVFLGLPFNI_P) as the template and primers, PosCTRL_BB_F and tRBSdX_BB_R (See, FIG. 14; Sequence Listing). The vector was then assessed by 0.8% agarose (0.5×TBE) gel electrophoresis and purified as described above. The mCherry insert was made by PCR using primers, mCherry-tRBSdX-F and mCherry-posCTRL_R, assessed by 0.8% agarose (0.5×TBE) gel electrophoresis, and purified as described above. The linearized vector and the insert were ligated using HiFi 2× Gibson Master Mix (NEB) according to manufacturer's protocol and transformed into chemically competent 10 beta cells as described above.

For mCherry-containing pBAD-tdTEVDB. pBAD-tdTEVDB was linearized by PCR using primers, TEV-DB-BB-F and TEV-DB-BB-R (see FIG. 14; Sequence Listing), assessed by 0.8% agarose (0.5×TBE) gel electrophoresis, and purified as described above. The mCherry insert was made by PCR using primers, mCherry-TEV-DB-F and mCherry-R (See, FIG. 14; Sequence Listing), assessed by 0.8% agarose (0.5×TBE) gel electrophoresis, and purified as described above. The linearized vector and the insert were ligated using HiFi 2× Gibson Master Mix (NEB) according to manufacturer's protocol and transformed into chemically competent 10 beta cells as described above. The two new constructs were sequence verified, before transformed into BL21 (DE3)+pRK793 cells, and assessed using the fluorescence assay method described below in Dual-Fluorescence Ratiometry Assay.

### Dual-Fluorescence Ratiometry Assay

1 μL of a set of pBAD plasmids (Negative control, pBAD-sfGF52-DVFLGLPFNI-P-mCherry, pBAD-tdTEVDB, and pBAD-tdTEVDB-mCherry) were transformed into BL21+pRK793 as described above. 40 μL of the transformant was spread out on selective plates consisting of LB Agar supplemented with 100 μg/mL ampicillin and 15 μg/mL tetracycline using coli rollers. After ˜16 h incubation at 37° C., three separate colonies were inoculated into 3×200 μL LB supplemented with 100 μg/mL ampicillin and 15 μg/mL tetracycline in a clear-bottom 96-well plate (Costar). After inoculation, the plate was sealed with parafilm and incubated overnight (˜16 h) at 37° C. with agitation (220 rpm), and these created the biological triplicates used for fluorescence measurements. The final OD₆₀₀ were measured for the overnight cultures using a plate reader instrument (SpectraMax iD3 from Molecular Devices) and were used to subculture down to a starting OD₆₀₀ of 0.05 in 200 μL of induction media (LB supplemented with 15 μg/mL tetracycline, 100 μg/mL ampicillin, 0.1 mM IPTG, 0.2% arabinose, and 20 mM Mg²⁺) in a clear-bottom 96-well plate. 96-well plates containing biological triplicates was loaded into an iD3 microplate reader pre-incubated at 30° C. and measured for both growth (OD₆₀₀ by absorbance) and GFP and mCherry fluorescence (GFP: excitation at 488 nm, emission at 535 nm; mCherry: excitation at 588 nm, emission at 650 nm) every 10 min over 16 h. The rest of the setting, including PMT and the speed of orbital mixing, stayed the same.

To obtain the ratio of the native fluorescence of GFP and mCherry, the corresponding backgrounds (GFP and mCherry signals from the negative control) were subtracted from the fluorescence signals (GFP and mCherry) generated from the positive control (pBAD-sfGF52-DVFLGLPFNI-P-mCherry). For the positive control, GFP generated signals that were 54% the level of mCherry (See, FIG. 4D). This served as a normalization factor. For pBAD-tdTEVDB-mCherry, the corresponding backgrounds were subtracted from each of the fluorescence signals. The background-subtracted GFP signals were divided by the background-subtracted mCherry signals, and then divided by the normalization factor (0.54) to generate the normalized GFP/mCherry ratio, indicating circularization efficiency.

### Library Generation

To improve ribosomal initiation on the circular mRNA, error-prone PCR (epPCR) was performed on the initiation region of pBAD-tdTEVDB plasmid that to install 1 or 2 point mutations on the target region. The starting point for these mutations was the pBAD-tdTEVDB variant with the trimmed-down context regions (DVFLG deleted from the 5′-exon context and PFNI deleted from the 3′-exon context, See, FIG. 5). The commercial GeneMorph II Random Mutagenesis kit (Agilent) that was used to perform epPCR, but using a modified protocol that involved performing several iterations of dilution and reamplification with a touchdown PCR protocol (51). The forward and reverse primers SD_lib_insert_F/R (see Sequence Listing and FIG. 14) were designed to span the initiation sequence. In the first round of epPCR. the manufacturer's protocol was adopted except that template plasmid was first diluted 109-fold and only 1 attogram (ag; 1 ag=10⁻¹⁸ g) of plasmid DNA was used as template in the PCR. The first epPCR product (primary amplicon) was size-verified by 1.2% agarose gel and diluted 1000-fold using Millipore water, and 0.5 μL of the diluent (ca. 2 pg) was used to seed a reamplification epPCR in a 25 μL scale that otherwise had the same primer, polymerase, and dNTP concentrations. Unlike the first epPCR, the reamplification PCR used a Touchdown protocol in which the annealing temperature started at 65° C. in the first cycle and decreased by 0.5° C. in each cycle. Under these optimal reaction condition found, the reamplification was repeated a total of nine times, in each case diluting the product by 1000-fold and using 0.5 μL of the diluent to seed the next PCR. After the final reamplification, the PCR products were subjected to a digest with DpnI and column-purified. Separately, backbone fragments were amplified using the primers SD_lib_BB_F/R (See, Sequence Listing and FIG. 14) with a high-fidelity DNA polymerase (Q5 polymerase, NEB) that were designed to have homology arms with the flanks of the mutagenized insert. The insert was then cloned into the backbone via In-Fusion cloning (TaKaRa Bio Inc.), and the ligated products were transformed into chemically competent 10 beta cells as described previously. The transformant was directly inoculated into liquid culture, from which plasmid DNA was purified via midi-prep to generate a plasmid library.

### Directed Evolution

1 μL of the plasmid library was transformed into 25 μL of BL21 (DE3)+pRK793 competent cells as previously described, and 40 μL of the transformant was spread over selective plates of LB agar supplemented with 15 μg/mL tetracycline and 100 μg/mL ampicillin using coli rollers: 4 separate plates were prepared for the first round of directed evolution, and 8 plates were prepared for the second round of directed evolution. As a reference sample, pBAD-tdTEVDB was also transformed into 25 μL of BL21 (DE3)+pRK793 competent cells as previously described. After ˜16 h incubation at 37° C., three colonies of pBAD-tdTEVDB were manually picked from the plate using sterile pipette tips and were inoculated into the first three wells (A1-3) of each of the four Nunc 96-Well Polypropylene DeepWell plates (2 mL well capacity; Fisher Scientific); each well contained 1.2 mL of LB media supplemented with 15 μg/mL tetracycline and 100 μg/mL ampicillin. The colonies on the plasmid library plates were automatically picked by sterilized needles of the RapidPick Colony Picking System (Wagner Life Science) through its corresponding software (Picker 5.0.1); 372 colonies were picked for the first round of directed evolution and 720 colonies were picked for the second round. The picked colonies were inoculated into the identical selective media in each of the remaining wells of the 96 DeepWell plates. After inoculation, the 96 DeepWell plates were covered with breathable sheets [Breathe-EASIER 6″×3.25″ (Cat. No. BERM-2000); Diversified BioTech] and incubated at 37° C. overnight with agitation (220 rpm). After ˜16 h incubation, 5 μL of each overnight culture was inoculated into 200 μL of LB supplemented 15 μg/mL tetracycline. 100 μg/mL ampicillin. 0.1 mM IPTG, 0.2% arabinose, and 20 mM MgCl₂ on black flat-bottom 96-well plates [4 plates; Caplugs Evergreen Labware Products (Cat. No. 290-8195-Z1F)] using a 20 μL multi-channel pipette (Eppendorf). The inoculated cultures in the black 96 well plates were again covered with breathable sheets and incubated at 30° C. with agitation (300 rpm) for 13 h (the 13-h time point was selected for measuring fluorescent signals based on the signal curve patterns of constructs observed up to that point). At the 13 h time point, all plates were taken out from the shaker and directly screened for fluorescent signals (excitation at 488 nm; emission at 535 nm; without lid) by Spark multimode plate reader (Tecan). Top-performing constructs were chosen and directly inoculated into LB supplemented with 15 μg/mL tetracycline and 100 μg/mL ampicillin, from which constructs were purified via mini-prep and sent off for Sanger sequencing. Once sequence-verified, 1 μL of each of the purified constructs were rephenotyped by transformation into 25 μL BL21 (DE3)+pRK793 competent cells and used to set up a fluorescence assay in biological triplicates.

### Additional Data

### Cell Growth Curve

To test for possible toxicities associated with loopable translation, three constructs corresponding to negative control, positive control, and pBAD-tdTEVDB were transformed into BL21+pRK793 cells (with TEV protease) and grown as described in the main text. Three separate colonies were inoculated into 3×200 μL of LB media supplemented with ampicillin, tetracycline, 0.2% arabinose, and 20 mM Mg²⁺ in a clear-bottom 96-well plate (Costar) and expressed at 30° C. With the starting OD₆₀₀ of 0.05, the growth was measured in every 10 min over the course of 16 hours (See, FIG. 11). No significant difference in growth rates across these three strains was found.

### Northern Blot

The negative control (pBAD-sfGFP₁₋₅₂) and pBAD-tdTEVDB were transformed into BL21 and expressed at 30° C. in LB media supplemented with Tetracycline, 0.2% arabinose, and 20 mM MgCl₂. With a starting OD₆₀₀ of 0.05, the cell cultures were grown to OD₆₀₀ of 1.0˜1.2. and centrifuged at 5,000 rpm for 15 min at 4° C. to generate cell pellets. Total RNAs were extracted from the cell pellets via Trizol extraction methods followed by the manufacturer's protocol (Invitrogen). The extracted RNAs were resuspended in 50 μL of DEPC-treated water and measured for concentrations using NanoDrop.

Approximately 3 μg of the extracted RNAs were mixed with 9 μL of 2× of RNA dye (NEB) via vortexing, denatured at 70° C. for 20 min, and separated by native electrophoresis with a 4% TBE-acrylamide gel using 0.5×TBE buffer at 180 V for 45 min at room temperature. The separated RNAs were electroblotted to a nylon membrane using iBlot2 (7 min; 20 V) and immobilized using “Auto Cross Link” mode of UV Stratalinker 1800 (Strategen). The fixed nylon blot was preincubated in a pre-heated hybridization buffer (UltraHyb buffer; Invitrogen) for 30 min at 42° C. Then a biotinylated DNA probe which was designed to anneal to RNA sequences corresponding to GFP residue 58 to 66 (present in the circularized loop generated by pBAD-tdTEVDB; see Sequence Listing) was added to the hybridization buffer to a final concentration of 100 pM, and the blot was hybridized overnight at 42° C. The blot was washed 2×5 min in Ambion NorthernMax™ Low Stringency Wash Buffer #1 (Invitrogen) and again washed 2×15 min in Ambion NorthernMax™ High Stringency Wash Buffer #2 (Invitrogen).

To block the nylon blot, the blot was incubated in 16 mL of 1×I-Block blocking buffer (1×PBS, 0.5% SDS, 0.1% I-Block Protein-Based Blocking Reagent (Invitrogen)) for 15 min at room temperature with a gentle shaking. Streptavidin-Horseradish Peroxide conjugate (Thermo Scientific) was added to the blocking buffer to a final concentration of 0.3 ug/mL, and the blot was incubated for 15 min with a gentle shaking. Then the nylon membrane was washed 4×5 min each with 20 mL of 1× wash buffer (1×PBS, 0.5% SDS) with a gentle shaking. After the washing, the nylon blot was incubated for 5 min in 8 mL mixture of SuperSignal™ West Pico Plus Chemiluminescent Substrates (mixed in a 1:1 ratio; Thermo Scientific) and taken image using ChemiDoc Touch Imaging System (BioRad).

Creating Constructs with Tandem Dragline Silk Multimers

A minigene plasmid (entitled “pIDT-silk-unit”) containing a flexible tag, part of MaSp1 sequence (dragline silk), and a His (6)-tag (see Sequence Listing) was ordered from IDT. and the above sequence of interest was amplified via PCR using primers Amp_silk_repetitive_unit_F and Gib_silk_tandem_seq_R (see Sequence Listing). The resulting PCR product has a recognition site for NheI between the flexible tag and the MaSp1 and a recognition site for SpeI between the MaSp1 and the His-tag, enabling a directional cloning in the downstream synthesis of constructs for tandem silk multimers. The PCR product was checked on 0.8% agarose gel. DpnI digested, and column-purified as previously described, and digested with NheI and SpeI. Then the silk insert was ligated back in a head-to-tail fashion into pIDT-silk-unit plasmid that was digested only with either NheI or SpeI (See, FIG. 1).^{25,39} A successful ligation removes the recognition site between the two silk-mers, generating a plasmid that has a tandem silk dimer.^{25,39} The above strategy was used to make three pIDT constructs containing tandem silk multimers in different lengths (silk 8-mer, 16-mer, and 24-mer). The synthesized silk multimers were excised from the above pIDT constructs using NheI and SpeI. separated by 0.8% agarose gel, and gel-purified using QIAquick Gel Extraction Kit (QIAGEN). For non-looped silk multimer constructs, pBAD-sfGFP plasmid was used as a template to generate a linearized pBAD backbone by PCR using linearize F/R (see Sequence Listing). Following PCR, the linearized pBAD backbone obtained recognition sites for AvrII and SpeI at the 5′ and 3′ end, respectively. The pBAD backbone was first digested with AvrII and SpeI, dephosphorylated, and then ligated with each of the silk multimer inserts that were digested with NheI and SpeI.^{25,39} For looped silk multimer constructs, the pBAD backbone was generated by PCR with primers TEVDB_Pemuted_F/R (see Sequence Listing) using pBAD-tdTEVDB with only 6 exonic context sequence on its 3′ end (See, FIG. 5E) as a template. The linearized pBAD backbone also has the recognition sites for AvrII and SpeI at each end, then was digested with AvrII and SpeI, dephosphorylated, and then ligated with each of the silk multimer inserts that were digested with NheI and SpeI. All six ligated products were transformed into 10-beta competent cells, and processed as described previously, resulting in three non-looped pBAD constructs and three looped pBAD constructs containing silk 8-mer, 16-mer, and 24-mer (See, FIG. 7A). The synthesized six constructs were verified by Sanger Sequencing.

### Protein Expression and Western Blot of Silk Constructs

For expression, the six constructs with silk multimers were transformed into BL21 (DE3) as previously described, and 40 μL of the transformants were spread out on selective plates with LB agar supplemented with 15 μg/ml tetracycline. The plates were incubated at 37° C. overnight (˜16h), and one colony of each plate was picked with sterile pipette tips and inoculated in 5 mL of LB supplemented with 15 μg/ml tetracycline to make overnight cultures which were incubated at 37° C. overnight with agitation (220 rpm). The overnight cultures were then used to start day cultures in 50 mL of LB supplemented with 7.5 μg/ml tetracycline, 0.2% arabinose and 20 mM MgCl₂ at a starting OD₆₀₀ of 0.05. The day cultures were incubated at 30° C. overnight with agitation (220 rpm), and cells from 1 mL aliquots were collected by centrifugation at 4° C. at 3000 g for 20 min and stored at −20° C. until future use.

Frozen cell pellets were thawed on ice for 15 min, resuspended with 500 μL of lysis buffer (1 mM Tris-HCl, pH 8.0, 20 mM NaH₂PO₄, 8 M Urea, 2 M thiourea; Table 1), and incubated at room temperature overnight (˜16h) with inversion (50 rpm, Roto-mini rotator). 24 μL of the resulting lysates were mixed with 6 μL of 5× Tris-Glycine-SDS loading buffer via vortexing. The samples were not heated and directly loaded onto pre-cast Novex WedgeWell 8% Tris-glycine gels with 3 μL of Pre-stained PAGE ruler as the ladder (ThermoFisher Scientific; #26619). The rest of the western was performed as previously described, using diluted anti-His primary antibody (Invitrogen) and anti-mouse-HRP secondary antibody (Invitrogen). The incubated membrane was rinsed as previously described, developed in chemiluminescence reagents (Signal West Femto Maximum Sensitive Substrate; ThermoFisher Scientific), and images were acquired using a Chemi-Doc imager (BioRad).

TABLE 1
Buffer Compositions. Composition of the buffers that
were used in Western blot for silk constructs.
10× Buffer A

| | Final Concentration (mM) |
|---|---|
| Trsi Base | 10 |
| 5M HCl | 5 |
| HaH₂PO₄ | 200 |

Lysis Buffer (5 mL)

| | |
|---|---|
| Amount | Final Concentration (M) |

| | | | |
|---|---|---|---|
| Urea | 2.4 | g | 8 |
| Thiourea | 0.75 | g | 2 |
| 10× buffer A | 500 | uL | 1× |

### REFERENCES

All publications, patent applications, patents, and other references mentioned in the specification are indicative of the level of those skilled in the art to which the presently disclosed subject matter pertains. All publications, patent applications, patents. and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Although the foregoing subject matter has been described in some detail by way of illustration and example for purposes of clarity of understanding, it will be understood by those skilled in the art that certain changes and modifications can be practiced within the scope of the appended claims.
1 Xia, X. X.; Qian, Z. G.; Ki, C. S.; Park, Y. H.; Kaplan, D. L.; Lee, S. Y. Native-Sized Recombinant Spider Silk Protein Produced in Metabolically Engineered Escherichia Coli Results in a Strong Fiber. Proc. Natl. Acad. Sci. U.S.A 2010, 107 (32), 14059-14063. https://doi.org/10.1073/pnas.1003366107.
2 Bowen, C. H.: Dai, B.; Sargent, C. J.; Bai, W.; Ladiwala, P.; Feng. H.; Huang, W.; Kaplan, D. L.; Galazka, J. M.; Zhang. F. Recombinant Spidroins Fully Replicate Primary Mechanical Properties of Natural Spider Silk. Biomacromolecules 2018, 19 (9), 3853-3860. https://doi.org/10.1021/acs.biomac.8b00980.
3 Rising. A.; Johansson, J. Toward Spinning Artificial Spider Silk. Nat. Chem. Biol. 2015, II (MAY), 309-315. https://doi.org/10.1038/nchembio.1789.
4 Omenetto. F. G.; Kaplan, D. L. New Opportunities for an Ancient Material. Science. 2010, 329 (5991), 528-531. https://doi.org/10.1126/science.1188936. New.
5 Andersson, M.; Jia, Q.; Abella, A.; Lec. X. Y.: Landrch, M.; Purhonen, P.; Hebert, H.; Tenje, M.; Robinson, C. V.; Meng, Q.; Plaza, G. R.; Johansson, J.; Rising, A. Biomimetic Spinning of Artificial Spider Silk from a Chimeric Minispidroin. Nat. Chem. Biol. 2017, 13 (3), 262-264. https://doi.org/10.1038/nchembio.2269.
6 Prince, J. T.; McGrath, K. P.; DiGirolamo, C. M.; Kaplan, D. L. Construction, Cloning, and Expression of Synthetic Genes Encoding Spider Dragline Silk. Biochemistry 1995, 34 (34). 10879-10885. https://doi.org/10.1021/bi00034a022.
7 Vacanti, J. P.; Langer, R. Tissue Engineering: The Design and Fabrication of Living Replacement Devices for Surgical Reconstruction and Transplantation. Lancet 1999, 354 (Special), S32-S34. https://doi.org/10.1016/S0140-6736 (99) 90247-7.
8 Paramonov. S. E.; Gauba. V.; Hartgerink. J. D. Synthesis of Collagen-like Peptide Polymers by Native Chemical Ligation. Macromolecules 2005, 38 (18). 7555-7561. https://doi.org/10.1021/ma0514065.
9 Shoulders. M. D.; Raines. R. T. Collagen Structure and Stability. Annu. Rev. Biochem. 2009, 78, 929-958. https://doi.org/10.1146/annurev.biochem.77.032207.120833.
10 Barnhart, M. M.; Chapman, M. R. Curli Biogenesis and Function. Annu. Rev. Microbiol. 2010, 60, 131-147. https://doi.org/10.1146/annurev.micro.60.080805.142106. Curli.
11 Abdali, Z.; Aminzare, M.: Zhu, X.; Debenedictis, E.; Xie, O.; Keten, S.: Dorval Courchesne. N. M. Curli-Mediated Self-Assembly of a Fibrous Protein Scaffold for Hydroxyapatite Mineralization. ACS Synth. Biol. 2020, 9 (12). 3334-3343. https://doi.org/10.1021/acssynbio.0c00415.
12 Dichl. A.; Roskc. Y.; Ball. L.; Chowdhury. A.; Hiller. M.; Molière, N.; Kramer. R.; Stoppler, D.; Worth, C. L.; Schlegel, B.; Leidert, M.; Cremer, N.; Erdmann, N.; Lopez, D.; Stephanowitz. H.; Krause. E.; van Rossum. B. J.; Schmieder. P.; Heinemann. U.; Turgay, K.; Akbey. Ü.; Oschkinat. H. Structural Changes of TasA in Biofilm Formation of Bacillus Subtilis. Proc. Natl. Acad. Sci. U.S.A 2018, 115 (13). 3237-3242. https://doi.org/10.1073/pnas.1718102115.
13 Knowles, T. P. J.; Buehler. M. J. Nanomechanics of Functional and Pathological Amyloid Materials. Nat. Nanotechnol. 2011, 6 (8). 469-479. https://doi.org/10.1038/nnano.2011.102.
14 Vural. M.; Lei. Y.; Pena-Francesch. A.: Jung. H.; Allen. B.; Terrones. M.; Demirel. M. C. Programmable Molecular Composites of Tandem Proteins with Graphene Oxide for Efficient Bimorph Actuators. Carbon N. Y. 2017, 118, 404-412. https://doi.org/10.1016/j.carbon.2017.03.053.
15 Pena-Francesch. A.; Akgun. B.; Miserez. A.; Zhu. W.; Gao, H.; Demirel. M. C. Pressure Sensitive Adhesion of an Elastomeric Protein Complex Extracted from Squid Ring Tecth. Adv. Funct. Mater. 2014, 24 (39). 6227-6233. https://doi.org/10.1002/adfm.201401534.
16 Pena-Francesch, A.; Jung. H.; Segad. M.; Colby, R. H.; Allen. B. D.; Demirel, M. C. Mechanical Properties of Tandem-Repeat Proteins Are Governed by Network Defects. ACS Biomater. Sci. Eng. 2018, 4 (3). 884-891. https://doi.org/10.1021/acsbiomaterials.7b00830.
17 Balc. J. B.; Gonen. S.; Liu. Y.; Shedffler. W.; King. N. P.; Baker. D. Accurate Design of Megadalton-Scale Two-Component Icosahedral Protein Complexes. Science (80-.). 2016, 353 (6297), 389-395. https://doi.org/10.5061/dryad.8c65s.
18 King. N. P.; Jacobitz. A. W.; Sawaya. M. R.; Goldschmidt. L.; Yeates. T. O. Structure and Folding of a Designed Knotted Protein. Proc. Natl. Acad. Sci. U.S.A 2010, 107 (48), 20732-20737. https://doi.org/10.1073/pnas. 1007602107.
19 Hershewe. J. M.; Wiseman. W. D.; Kath. J. E.; Buck. C. C.; Gupta. M. K.; Dennis. P. B.; Naik, R. R.; Jewett, M. C. Characterizing and Controlling Nanoscale Self-Assembly of Suckerin-12. ACS Synth. Biol. 2020, 9 (12). 3388-3399. https://doi.org/10.1021/acssynbio.0c00442.
20 Votteler. J.; Ogohara. C.; Yi. S.; Hsia, Y.: Nattermann. U.; Belnap. D. M.; King. N. P.; Sundquist. W. I. Designed Proteins Induce the Formation of Nanocage-Containing Extracellular Vesicles. Nature 2016, 540 (7632), 292-295. https://doi.org/10.1038/nature20607.
21 Jung. H.; Pena-Francesch. A.; Saadat, A.: Sebastian, A.; Kim, D. H.; Hamilton, R. F.; Albert. I.; Allen. B. D.; Demirel. M. C. Molecular Tandem Repeat Strategy for Elucidating Mechanical Properties of High-Strength Proteins. Proc. Natl. Acad. Sci. U.S.A 2016, 113 (23). 6478-6483. https://doi.org/10.1073/pnas.1521645113.
22 Ford. E.: Ares Jr., M. Synthesis of Circular RNA in Bacteria and Yeast Using RNA Cyclase Ribozymes Derived from a Group I Intron of Phage T4. Proc. Natl. Acad. Sci. USA 1994, 91 (8), 3117-3121.
23 Perriman. R.; Ares. M. Circular MRNA Can Direct Translation of Extremely Long Repeating-Sequence Proteins in Vivo. Rna 1998, 4 (9), 1047-1054. https://doi.org/10.1017/S135583829898061X.
24 Perriman. R. Circular MRNA Encoding for Monomeric and Polymeric Green Fluorescent Protein. Methods Mol. Biol. 2001, 183, 69-85. https://doi.org/10.1385/1-59259-280-5:069.
25 Shub. D. A.; Gott. J. M.; Xu. M. Q.: Lang. B. F.: Michel. F.; Tomaschewski. J.; Pedersen-Lanc, J.; Belfort, M. Structural Conservation among Three Homologous Introns of Bacteriophage T4 and the Group I Introns of Eukaryotes. Proc. Natl. Acad. Sci. U.S.A 1988, 85 (4). 1151-1155. https://doi.org/10.1073/pnas.85.4.1151.
26 Umckage. S.; Uchara. T.: Fujita. Y.; Suzuki, H.; Kikuchi. Y. In Vivo Circular RNA Expression by the Permuted Intron-Exon Method. Innov. Biotechnol. 2012, 108 (4). 354-356. https://doi.org/10.5772/28220.
27 Sprengart. M. L.; Fuchs. E.; Porter. A. G. The Downstream Box: An Efficient and Independent Translation Initiation Signal in Escherichia Coli. EMBO J. 1996, 15 (3). 665-674. https://doi.org/10.1002/j.1460-2075.1996.tb00399.x.
28 Etchegaray. J.-P.: Inouye. M. Translational Enhancement by an Element Downstream of the Initiation Codon in Escherichia Coli *; 1999; Vol. 274.
29 Van Den Berg. S.; Löfdahl. P. A.; Härd. T.; Berglund. H. Improved Solubility of TEV Protease by Directed Evolution. J. Biotechnol. 2006, 121 (3). 291-298. https://doi.org/10.1016/j.jbiotec.2005.08.006.
30 Kapust. R. B.; Tözsér. J.; Fox. J. D.; Anderson. D. E.; Cherry. S.; Copeland. T. D.; Waugh. D. S. Tobacco Etch Virus Protease: Mechanism of Autolysis and Rational Design of Stable Mutants with Wild-Type Catalytic Proficiency. Protein Eng. 2001, 14 (12). 993-1000. https://doi.org/10.1093/protein/14.12.993.
31 Cech. T. R. Self-Splicing of Group I Introns. Ann. Rev. Biochem 1990, 59, 543-568. https://doi.org/10.1146/annurev.bi.59.070190.002551.
32 Guo. F.; Gooding. A. R.; Cech. T. R. Structure of the Tetrahymena Ribozyme. Mol. Cell 2004, 16 (3), 351-362. https://doi.org/10.1016/j.molcel.2004.10.003.
33 Michel. F.; Hanna. M.; Green. R.; Bartel. D. P.; Szostak. J. W. The Guanosine Binding Site of the Tetrahymena Ribozyme. Nature 1989, 342 (6248), 391-395. https://doi.org/10.1038/342391a0.
34 Legault. P.; Herschlag. D.; Celander. D. W.; Cech. T. R. Mutations at the Guanosine-Binding Site of the Tetrahymena Ribozyme Also Affect Site-Specific Hydrolysis. Nucleic Acids Res. 1992, 20 (24). 6613-6619. https://doi.org/10.1093/nar/20.24.6613.
35 Varik, V.; Oliveira, S. R. A.; Hauryliuk, V.; Tenson, T. HPLC-Based Quantification of Bacterial Housekeeping Nucleotides and Alarmone Messengers PpGpp and PppGpp. Sci. Rep. 2017, 7 (1). 1-12. https://doi.org/10.1038/s41598-017-10988-6.
36 Chen, X.; Mohr. G.; Lambowitz, A. M. The Neurosporea Crassa CYT-18 Protein C-Terminal RNA-Binding Domain Helps Stabilize Interdomain Tertiary Interactions in Group I Introns. Rna 2004, 10 (4), 634-644. https://doi.org/10.1261/rna.5212604.
37 Wci. L.; Cai. X.; Qi. Z.; Rong. L.; Cheng. B.; Fan. J. In Vivo and in Vitro Characterization of TEV Protease Mutants. Protein Expr. Purif. 2012, 83 (2). 157-163. https://doi.org/10.1016/j.pep.2012.03.011.
38 Fang. J.; Chen. L.; Cheng. B.; Fan. J. Engineering Soluble Tobacco Etch Virus Protease Accompanies the Loss of Stability. Protein Expr. Purif. 2013, 92 (1), 29-35. https://doi.org/10.1016/j.pep.2013.08.015.
39 Sanchez. M. I.; Ting. A. Y. Directed Evolution Improves the Catalytic Efficiency of TEV Protease. Nat. Methods 2020, 17 (2), 167-174. https://doi.org/10.1038/s41592-019-0665-7.
40 Colussi. T. M.; Costantino. D. A.; Zhu. J.; Donohue. J. P.; Korostelev. A. A.; Jaafar. Z. A.; Plank, T. D. M.; Noller, H. F.; Kieft, J. S. Initiation of Translation in Bacteria by a Structured Eukaryotic IRES RNA. Nature 2015, 519 (7541), 110-113. https://doi.org/10.1038/nature14219.
41 Litke. J. L.; Jaffrey. S. R. Highly Efficient Expression of Circular RNA Aptamers in Cells Using Autocatalytic Transcripts. Nat. Biotechnol. 2019, 37 (6). 667-675. https://doi.org/10.1038/s41587-019-0090-6.
42 Levin-Karp. A.; Barenholz. U.; Barcia. T.; Dayagi. M.; Zelcbuch. L.; Antonovsky. N.; Noor, E.; Milo, R. Quantifying Translational Coupling in E. coli Synthetic Operons Using RBS Modulation and Fluorescent Reporters. ACS Synth. Biol. 2013, 2 (6). 327-336. https://doi.org/10.1021/sb400002n.
43 Zhang. Y.; Chen. H.; Zhang. Y.; Yin. H.: Zhou. C.; Wang. Y. Direct RBS Engineering of the Biosynthetic Gene Cluster for Efficient Productivity of Violaceins in E. coli. Microb. Cell Fact. 2021, 20 (1). 1-13. https://doi.org/10.1186/s12934-021-01518-1.
44 Zhang. X.; Lin. Y.; Wu. Q.; Wang. Y.; Chen. G. Q. Synthetic Biology and Genome-Editing Tools for Improving PHA Metabolic Engineering. Trends Biotechnol. 2020, 38 (7), 689-700. https://doi.org/10.1016/j.tibtech.2019.10.006.
45 Sørensen. M. A.; Fricke. J.; Pedersen, S. Ribosomal Protein S1 Is Required for Translation of Most, It Not All. Natural MRNAs in Escherichia Coli in Vivo. J. Mol. Biol. 1998, 280 (4). 561-569. https://doi.org/10.1006/jmbi.1998.1909.
46 Boni. I. V.; Lsacva. D. M.; Musychenko, M. L.; Tzareva. N. V. Ribosome-Messenger Recognition: MRNA Target Sites for Ribosomal Protein S1. Nucleic Acids Res. 1991, 19 (1). 155-162. https://doi.org/10.1093/nar/19.1.155.
47 Rasila. T. S.; Pajunen. M. I.; Savilahti. H. Critical Evaluation of Random Mutagenesis by Error-Prone Polymerase Chain Reaction Protocols, Escherichia Coli Mutator Strain, and Hydroxylamine Treatment. Anal. Biochem. 2009, 388 (1). 71-80. https://doi.org/10.1016/j.ab.2009.02.008.
48 McInerney. P.; Adams. P.; Hadi. M. Z. Error Rate Comparison during Polymerase Chain Reaction by DNA Polymerase. Mol. Biol. Int. 2014, 2014, 1-8. https://doi.org/10.1155/2014/287430.
49 Patrick. W. M.; Firth. A. E.; Blackburn. J. M. User-Friendly Algorithms for Estimating Completeness and Diversity in Randomized Protein-Encoding Libraries. Protein Eng. 2003, 16 (6). 451-457. https://doi.org/10.1093/protein/gzg057.
50 Vanhercke, T.; Ampe. C.; Tirry. L.; Denolf, P. Reducing Mutational Bias in Random Protein Libraries. Anal. Biochem. 2005, 339 (1). 9-14. https://doi.org/10.1016/j.ab.2004.11.032.
51 Lec. S. O.; Fried. S. D. An Error-Prone PCR Method for Small Amplicons. Anal. Biochem. 2021.
52 Farabaugh, P. J.; Björk, G. R. How Translational Accuracy Influences Reading Frame Maintenance. EMBO J. 1999, 18 (6). 1427-1434. https://doi.org/10.1093/emboj/18.6.1427.
53 Sarr. M.; Kronqvist, N.; Chen, G.; Aleksis, R.; Purhonen, P.; Hebert, H.; Jaudzems, K.; Rising, A.; Johansson, J. A Spidroin-Derived Solubility Tag Enables Controlled Aggregation of a Designed Amyloid Protein. FEBS J. 2018, 285 (10). 1873-1885. https://doi.org/10.1111/febs.14451.
54 Vincenz-Donnelly. L.; Holthusen. H.; Körner. R.; Hansen, E. C.; Presto. J.; Johansson. J.; Sawarkar. R.; Hartl. F. U.; Hipp. M. S. High Capacity of the Endoplasmic Reticulum to Prevent Secretion and Aggregation of Amyloidogenic Proteins. EMBO J. 2018, 37 (3), 337-350. https://doi.org/10.15252/embj.201695841.
55 Abelcin, A.; Chen, G.; Kitoka, K.; Aleksis, R.: Oleskovs, F.; Sarr, M.; Landreh, M.; Pahnke, J.; Nordling. K.: Kronqvist. N.; Jaudzems. K.; Rising. A.; Johansson. J.; Biverstål. H. High-Yield Production of Amyloid-β Peptide Enabled by a Customized Spider Silk Domain. Sci. Rep. 2020, 10 (1). 1-10. https://doi.org/10.1038/s41598-019-57143-x.
56 Landrch. M.; Andersson. M.; Marklund. E. G.; Jia. Q.; Meng. Q.; Johansson. J.; Robinson. C. V.; Rising. A. Mass Spectrometry Captures Structural Intermediates in Protein Fiber Self-Assembly. Chem. Commun. 2017, 53 (23). 3319-3322. https://doi.org/10.1039/c7cc00307b.
57 Jin. H. J.; Kaplan. D. L. Mechanism of Silk Processing in Insects and Spiders. Nature 2003, 424 (6952). 1057-1061. https://doi.org/10.1038/nature01809.
58 Schwarze, S.; Zwettler, F. U.; Johnson, C. M.; Neuweiler. H. The N-Terminal Domains of Spider Silk Proteins Assemble Ultrafast and Protected from Charge Screening. Nat. Commun. 2013, 4. https://doi.org/10.1038/ncomms3815.
59 Ittah. S.; Cohen, S.; Garty, S.; Cohn, D.; Gat, U. An Essential Role for the C-Terminal Domain of a Dragline Spider Silk Protein in Directing Fiber Formation. Biomacromolecules 2006, 7 (6). 1790-1795. https://doi.org/10.1021/bm060120k.
60 Nguyen. P. Q.; Courchesne. N.-M. D.; Duraj-Thatte. A.; Praveschotinunt. P.; Joshi. N. S. Engineering Living Materials: Prospects and Challenges for Using Biological Systems to Direct the Assembly of Smart Materials. Physiol. Behav. 2016, 176 (12), 139-148. https://doi.org/10.1002/adma.201704847. Engineered.
61 Chen. A. Y.; Deng. Z.; Billings. A. N.; Seker. U. O. S.; Lu. M. Y.; Citorik. R. J.; Zakeri. B.; Lu, T. K. Synthesis and Patterning of Tunable Multiscale Materials with Engineered Cells. Nat. Mater. 2014, 13 (5). 515-523. https://doi.org/10.1038/nmat3912.
62 Nguyen, P. Q.; Botyanszki, Z.; Tay, P. K. R.; Joshi, N. S. Programmable Biofilm-Based Materials from Engineered Curli Nanofibres. Nat. Commun. 2014, 5. 1-10. https://doi.org/10.1038/ncomms5945.
63 Duraj-Thatte, A. M.; Manjula-Basavanna, A.; Courchesne, N. M. D.; Cannici, G. I.; Sánchez-Ferrer. A.; Frank. B. P.; van't Hag. L.; Cotts. S. K.; Fairbrother. D. H.; Mezzenga. R.; Joshi, N. S. Water-Processable, Biodegradable and Coatable Aquaplastic from Engineered Biofilms. Nat. Chem. Biol. 2021. https://doi.org/10.1038/s41589-021-00773-y.
64 Dubey, G.; Mequanint, K. Conjugation of Fibronectin onto Three-Dimensional Porous Scaffolds for Vascular Tissue Engineering Applications. Acta Biomater. 2011, 7 (3), 1114-1125. https://doi.org/10.1016/j.actbio.2010.11.010.
65 Glowacki, J.; Mizuno, S. Collagen Scaffolds for Tissue Engineering. Biopolymers 2008, 89 (5), 338-344. https://doi.org/10.1002/bip.20871.
66 Boccaccini, A. R.; Blaker, J. J. Bioactive Composite Materials for Tissue Engineering Scaffolds. Rev. Med. Devices Expert 2005, 2 (3), 303-317. https://doi.org/10.1586/17434440.2.3.303.

What is claimed is:

1. A genetic construct for performing loopable translation, the construct comprising in the following order: (i) a 3′ portion of a Group I self-splicing intron; (ii) a C-terminal portion of a gene of interest, wherein the C-terminal portion of the gene of interest comprises a 3′-exonic context sequence at its 5′ end; (iii) a ribosome binding site (RBS); (iv) a N-terminal portion of the gene of interest, wherein the N-terminal portion of the gene of interest comprises a 5′-exonic sequence at its 3′ end; and (v) a 5′ portion of the Group I self-splicing intron.

2. The genetic construct of claim 1, wherein the genetic construct further comprises an initiator codon (AUG) and a downstream box (DB) sequence after the ribosome binding site (RBS).

3. The genetic construct of claim 1 or claim 2, wherein the genetic construct further comprises a TEV protease cleavage site at the end of the C-terminal portion of a gene of interest.

4. The genetic construct of any of claims 1-3, wherein the Group I self-splicing intron is a T4 bacteriophage thymidylate synthase (td) intron substituted with a Tetrahymena thermophila rRNA intron, Chlamydomonas reinhardii rRNA intron, or T4 bacteriophage sun Y intron substituted with a Tetrahymena thermophila rRNA intron.

5. The genetic construct of any of claims 1-4, wherein the 3′-exonic context sequence functions as an internal guide sequence (IGS) for site-specific splicing.

6. The genetic construct of any of claims 1-5, wherein the 5′-exonic context sequence functions as an internal guide sequence (IGS) for site-specific splicing.

7. The genetic construct of any of claims 1-6, wherein the gene of interest have a length of from 700 nucleotides to 1500 nucleotides.

8. The genetic construct of any of claims 1-7, wherein the gene of interest is a gene that encodes green fluorescent protein (GFP).

9. The genetic construct of claim 8, wherein the GFP gene has the sequence of SEQ ID NO: 1.

10. The genetic construct of claim 9, wherein the N-terminal portion of the GFP gene comprises nucleotides 1 to 52 of SEQ ID NO: 1 and the C-terminal portion of the GFP gene comprises nucleotides 53 to 241 of SEQ ID NO:1.

11. The genetic construct of claim 1-10, wherein the 5′-exon context sequence includes at least 1 codon, at least 2 codons, at least 3 codons, at least 4 codons, or at least 5 codons.

12. The genetic construct of any of claims 1-11, wherein the 5′ exon context sequence wherein at least one codon is GGU (glycine).

13. The genetic construct of claim 8-10, wherein the 5′-exon context sequence does not include a codon, but in which the N-terminal portion of the gene of interest ends with GGU (glycine).

14. The genetic construct of any of claims 1-13, wherein the 3′-exon context sequence includes at least 1 codon, at least 2 codons, at least 3 codons, at least 4 codons, or at least 5 codons.

15. The genetic construct of any of claims 1-14, wherein the 3′ exon context sequence wherein at least one codon is CUA (leucine).

16. The genetic construct of any of claims 1-15, wherein the gene of interest encodes a biofilm forming CsgA protein, dragline spidroin protein with soluble end-domains, a Squid Ring Teeth (SRT) protein, a collagen, or an mRNA-vaccine.

17. The genetic construct of any of claims 1-16, wherein the Group I self-splicing intron requires the use of at least two co-factors.

18. The genetic construct of claim 17, wherein the co-factors are a divalent cation and a guanosine nucleoside.

19. The genetic construct of claim 18, wherein the divalent cation is a magnesium cation.

20. The genetic construct of any of claims 17-19, wherein the divalent cation is used in an amount from about 1 to about 30 mM to increase construct performance.

21. The genetic construct of any of claims 1-20, wherein the construct is pBAD-tdTEVDB.

22. The genetic construct of any of claims 1-21, wherein the genetic construct contains a mutation in a 36-base pair region covering the RBS, a RBS spacer, an initiator codon, or a DB for increasing translational efficiency and product yield.

23. The genetic construct of claim 22, wherein the mutation is introduced using a modified error-prone PCR method.

24. The genetic construct of claim 22, wherein the initiator codon is an initiator methionine.

25. The genetic construct of claim 23, wherein the mutation is C-15A, G4C or C-15A/G4C.

26. A kit comprising:
a. the genetic construct of any of claims 1-25; and
b. at least two co-factors.

27. The kit of claim 26, wherein the co-factors are a divalent cation and a guanosine nucleoside.

28. The kit of claim 27, wherein the divalent cation is a magnesium cation.

29. A method of producing biomaterials, the method comprising the step of transforming a host cell with the genetic construct of any of claims 1-25, adding at least two co-factors, and culturing the host cell at a temperature of about 25° C. to about 37° C. to produce a biomaterial.

30. The method of claim 29, wherein the host cell is a bacterial cell, a yeast cell, or a mammalian cell.

31. The method of claim 29 or claim 30, wherein the genetic construct further comprises an inducible promoter.

32. The method of claim 31, wherein the inducible promoter is an Arabinose-inducible pBAD promoter or a GroES promoter.

33. The method of any of claims 29-32, wherein the host cell is a bacterial cell.

34. The method of claim 33, wherein the bacterial cell is an E. coli or a gram-positive bacterium.

35. The method of claim 34, wherein the gram-positive bacterium is Bacillus subtilis.

36. The method of any of claims 29-35, wherein the co-factors are a divalent cation and a guanosine nucleoside.

37. The method of claim 36, wherein the divalent cation is a magnesium cation.

38. The method of claim 37, wherein the magnesium cation is added to the culture in an amount of to about 1 to about 30 mM.

39. The method of any of claims 29-38, wherein the biomaterial is dragline silk comprising spidroins, a biofilm comprising curli proteins such as curli subunit A (CsgA), looped extracellular matrix (ECM) proteins such as fibronectin, laminin, collagen, reticulin, keratin, and elastin, squid ring teeth (SRT) proteins, globular cage-like protein nanomaterials, or any combinations thereof.


SEQUENCE LISTING (103 sequences)

SEQ ID NO: 1 - DNA, 750 bp, Synthetic construct
  source 1..750: mol_type = other DNA
atggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccat

SEQ ID NO: 2 - DNA, 42 bp, Synthetic construct
  source 1..42: mol_type = other DNA
aatttatttgcaccaccggttaaagctcgagcgaagcttggg

SEQ ID NO: 3 - DNA, 26 bp, Synthetic construct
  source 1..26: mol_type = other DNA
accggtggtgcaaataaatttcaggg

SEQ ID NO: 4 - DNA, 44 bp, Synthetic construct
  source 1..44: mol_type = other DNA
tttcttgggtctaccgtttaatattaaactgccggttccgtggc

SEQ ID NO: 5 - DNA, 50 bp, Synthetic construct
  source 1..50: mol_type = other DNA
taaacggtagacccaagaaaacatcaccggtggtgcaaataaatttcagg

SEQ ID NO: 6 - DNA, 22 bp, Synthetic construct
  source 1..22: mol_type = other DNA
gtctccccatgcgagagtaggg

SEQ ID NO: 7 - DNA, 33 bp, Synthetic construct
  source 1..33: mol_type = other DNA
atggagaaacagtagagagttgcgataaaaagc

SEQ ID NO: 8 - DNA, 45 bp, Synthetic construct
  source 1..45: mol_type = other DNA
gaaaacctgtattttcagggccaggaggaattaaccatggttagc

SEQ ID NO: 9 - DNA, 43 bp, Synthetic construct
  source 1..43: mol_type = other DNA
gccctgaaaatacaggttttcatggtgatgatgatggtggctg

SEQ ID NO: 10 - DNA, 55 bp, Synthetic construct
  source 1..55: mol_type = other DNA
aaggagatatacatatggctagcatgaatcacaaaggtgaagaactgtttaccgg

SEQ ID NO: 11 - DNA, 46 bp, Synthetic construct
  source 1..46: mol_type = other DNA
agccatatgtatatctccttggccctgaaaatacaggttttcatgg

SEQ ID NO: 12 - DNA, 29 bp, Synthetic construct
  source 1..29: mol_type = other DNA
taagaaaacctgtattttcagggccaagg

SEQ ID NO: 13 - DNA, 23 bp, Synthetic construct
  source 1..23: mol_type = other DNA
atggtgatgatgatggtggctgc

SEQ ID NO: 14 - DNA, 49 bp, Synthetic construct
  source 1..49: mol_type = other DNA
atttgcaccaccggtgttttcttgggttaattgaggcctgagtataagg

SEQ ID NO: 15 - DNA, 46 bp, Synthetic construct
  source 1..46: mol_type = other DNA
atttgcaccaccggtttcttgggttaattgaggcctgagtataagg

SEQ ID NO: 16 - DNA, 37 bp, Synthetic construct
  source 1..37: mol_type = other DNA
atttgcaccaccggttaattgaggcctgagtataagg

SEQ ID NO: 17 - DNA, 71 bp, Synthetic construct
  source 1..71: mol_type = other DNA
atttgcaccaccggttaattgaggccggtgtataaggtgacttatacttgtaatctatctaaacggggaac

SEQ ID NO: 18 - DNA, 25 bp, Synthetic construct
  source 1..25: mol_type = other DNA
accggtggtgcaaataaatttcagg

SEQ ID NO: 19 - DNA, 39 bp, Synthetic construct
  source 1..39: mol_type = other DNA
aacataatgctaccgtttaataaactgccggttccgtgg

SEQ ID NO: 20 - DNA, 36 bp, Synthetic construct
  source 1..36: mol_type = other DNA
aacataatgctaccgtttaaactgccggttccgtgg

SEQ ID NO: 21 - DNA, 33 bp, Synthetic construct
  source 1..33: mol_type = other DNA
aacataatgctaccgaaactgccggttccgtgg

SEQ ID NO: 22 - DNA, 36 bp, Synthetic construct
  source 1..36: mol_type = other DNA
tatctgaacataatgctaaaactgccggttccgtgg

SEQ ID NO: 23 - DNA, 34 bp, Synthetic construct
  source 1..34: mol_type = other DNA
tatctgaacataatgaaactgccggttccgtggc

SEQ ID NO: 24 - DNA, 36 bp, Synthetic construct
  source 1..36: mol_type = other DNA
tatctgaacataatgctcaaactgccggttccgtgg

SEQ ID NO: 25 - DNA, 33 bp, Synthetic construct
  source 1..33: mol_type = other DNA
cggtagcattatgttcagataaggtcgttaatc

SEQ ID NO: 26 - DNA, 33 bp, Synthetic construct
  source 1..33: mol_type = other DNA
cattatgttcagataaggtcgttaatcttaccc

SEQ ID NO: 27 - DNA, 33 bp, Synthetic construct
  source 1..33: mol_type = other DNA
ggccggccggccatgcatatccttagcgaaagc

SEQ ID NO: 28 - DNA, 39 bp, Synthetic construct
  source 1..39: mol_type = other DNA
gtagaaccggccggccggccaattcgaattcagcgtagc

SEQ ID NO: 29 - DNA, 36 bp, Synthetic construct
  source 1..36: mol_type = other DNA
ggccggccggccggttctacataaatgcctaacgac

SEQ ID NO: 30 - DNA, 41 bp, Synthetic construct
  source 1..41: mol_type = other DNA
atatgcatggccggccggccagtcctacaatttagcacggg

SEQ ID NO: 31 - DNA, 78 bp, Synthetic construct
  source 1..78: mol_type = other DNA
aacctgtattttcagggcggtggcagcggcggagggagtggtggacaaggagatatacatatggctagcatgaatcac

SEQ ID NO: 32 - DNA, 77 bp, Synthetic construct
  source 1..77: mol_type = other DNA
ctgaaaatacaggttttccccgccactaccgccaccgcttcctcctttatacagttcatccataccgtgggtaatgc

SEQ ID NO: 33 - DNA, 49 bp, Synthetic construct
  source 1..49: mol_type = other DNA
ccatgaaaacctgtattttcagggcattgctcgctcaaacattaagtgg

SEQ ID NO: 34 - DNA, 39 bp, Synthetic construct
  source 1..39: mol_type = other DNA
catggtaaattctttttcttgaagtgagattcttttcgc

SEQ ID NO: 35 - DNA, 50 bp, Synthetic construct
  source 1..50: mol_type = other DNA
acttcaagaaaaagaatttaccatgggtgaagaactgtttaccggcgttg

SEQ ID NO: 36 - DNA, 25 bp, Synthetic construct
  source 1..25: mol_type = other DNA
gccctgaaaatacaggttttcatgg

SEQ ID NO: 37 - DNA, 52 bp, Synthetic construct
  source 1..52: mol_type = other DNA
gggctaacaggaggaattaaccatgggcattgctcgctcaaacattaagtgg

SEQ ID NO: 38 - DNA, 32 bp, Synthetic construct
  source 1..32: mol_type = other DNA
catggttaattcctcctgttagcccaaaaaac

SEQ ID NO: 39 - DNA, 44 bp, Synthetic construct
  source 1..44: mol_type = other DNA
aaggagatacatatgaattacgaattactgaccactgaaaatgc

SEQ ID NO: 40 - DNA, 46 bp, Synthetic construct
  source 1..46: mol_type = other DNA
ggagtggtgaatccgttagcgattatccttttacgcacaccacctg

SEQ ID NO: 41 - DNA, 22 bp, Synthetic construct
  source 1..22: mol_type = other DNA
tcgctaacggattcaccactcc

SEQ ID NO: 42 - DNA, 40 bp, Synthetic construct
  source 1..40: mol_type = other DNA
ttcgtaattcatatgtatctccttcaggtcgaggtggccc

SEQ ID NO: 43 - DNA, 20 bp, Synthetic construct
  source 1..20: mol_type = other DNA
ggtgaagaactgtttaccgg

SEQ ID NO: 44 - DNA, 21 bp, Synthetic construct
  source 1..21: mol_type = other DNA
gccctgaaaatacaggttttc

SEQ ID NO: 45 - DNA, 21 bp, Synthetic construct
  source 1..21: mol_type = other DNA
gaaaacctgtattttcagggc

SEQ ID NO: 46 - DNA, 20 bp, Synthetic construct
  source 1..20: mol_type = other DNA
ccggtaaacagttcttcacc

SEQ ID NO: 47 - DNA, 21 bp, Synthetic construct
  source 1..21: mol_type = other DNA
gtctccccatgcgagagtagg

SEQ ID NO: 48 - DNA, 24 bp, Synthetic construct
  source 1..24: mol_type = other DNA
atggagaaacagtagagagttgcg

SEQ ID NO: 49 - DNA, 31 bp, Synthetic construct
  source 1..31: mol_type = other DNA
aatggaacactggtggtccaatcactacatg

SEQ ID NO: 50 - DNA, 35 bp, Synthetic construct
  source 1..35: mol_type = other DNA
atatgccatcacctgaagggaatgtgcaactagtg

SEQ ID NO: 51 - DNA, 33 bp, Synthetic construct
  source 1..33: mol_type = other DNA
cattcccttcaggtgatggcatattctggaagc

SEQ ID NO: 52 - DNA, 36 bp, Synthetic construct
  source 1..36: mol_type = other DNA
gaccaccagtgttccattatttcttctaaacaagtg

SEQ ID NO: 53 - DNA, 25 bp, Synthetic construct
  source 1..25: mol_type = other DNA
taaggatcctctagagtcgacctgc

SEQ ID NO: 54 - DNA, 30 bp, Synthetic construct
  source 1..30: mol_type = other DNA
aaacaagctttctccatgatgatgatgatg

SEQ ID NO: 55 - DNA, 17 bp, Synthetic construct
  source 1..17: mol_type = other DNA
ggcggtagtggcggttc

SEQ ID NO: 56 - DNA, 25 bp, Synthetic construct
  source 1..25: mol_type = other DNA
atggtgatgatgatggtgactagtc

SEQ ID NO: 57 - DNA, 57 bp, Synthetic construct
  source 1..57: mol_type = other DNA
aaaaaacctaggcaccatcatcatcaccacaccggttaattgaggcctgagtataag

SEQ ID NO: 58 - DNA, 74 bp, Synthetic construct
  source 1..74: mol_type = other DNA
aaaaaactagttttgtgattcatgctagccatatgtatatctccttgcggtagcattatgttcagataaggtcg

SEQ ID NO: 59 - DNA, 45 bp, Synthetic construct
  source 1..45: mol_type = other DNA
aaaaaacctaggcatcatcatcatcatcattgagtttaaacggtc

SEQ ID NO: 60 - DNA, 37 bp, Synthetic construct
  source 1..37: mol_type = other DNA
aaaaaaactagtcatggttaattcctcctgttagccc

SEQ ID NO: 61 - DNA, 22 bp, Synthetic construct
  source 1..22: mol_type = other DNA
tcgctaacggattcaccactcc

SEQ ID NO: 62 - DNA, 40 bp, Synthetic construct; identical to SEQ ID NO: 42
  source 1..40: mol_type = other DNA

SEQ ID NO: 63 - DNA, 52 bp, Synthetic construct
  source 1..52: mol_type = other DNA
gccgggccacctcgacctgaatgaattacgaattactgaccactgaaaatgc

SEQ ID NO: 64 - DNA, 44 bp, Synthetic construct
  source 1..44: mol_type = other DNA
gaggtgccgccggcttccatttatccttttacgcacaccacctg

SEQ ID NO: 65 - DNA, 26 bp, Synthetic construct
  source 1..26: mol_type = other DNA
ggtcagggtggtcaccagggtcggcc

SEQ ID NO: 66 - DNA, 21 bp, Synthetic construct
  source 1..21: mol_type = other DNA
taaagctcgagcgaagcttgg

SEQ ID NO: 67 - DNA, 43 bp, Synthetic construct
  source 1..43: mol_type = other DNA
ccctgaaaatacaggttttcatggtgatgatgatggtggctgc

SEQ ID NO: 68 - DNA, 40 bp, Synthetic construct
  source 1..40: mol_type = other DNA
gaaaacctgtattttcagggcatggtgagcaagggcgagg

SEQ ID NO: 69 - DNA, 40 bp, Synthetic construct
  source 1..40: mol_type = other DNA
aagcttcgctcgagctttacttgtacagctcgtccatgcc

SEQ ID NO: 70 - DNA, 55 bp, Synthetic construct
  source 1..55: mol_type = other DNA
ggcagcagcgagaatctttatttccaaggtggtgaagaactgtttaccggcgttg

SEQ ID NO: 71 - DNA, 33 bp, Synthetic construct
  source 1..33: mol_type = other DNA
tttgtgattcatgctagccatatgtatatctcc

SEQ ID NO: 72 - DNA, 39 bp, Synthetic construct
  source 1..39: mol_type = other DNA
tggctagcatgaatcacaaaatggtgagcaagggcgagg

SEQ ID NO: 73 - DNA, 51 bp, Synthetic construct
  source 1..51: mol_type = other DNA
ataaagattctcgctgctgccgctgctgcccttgtacagctcgtccatgcc

SEQ ID NO: 74 - DNA, 1199 bp, Synthetic construct
  source 1..1199: mol_type = other DNA
tcgcaactctctactgtttctccatacccgttttttgggctacgctgaattcgaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccattaattaaacggtctccagcttggctgttttggcggataacaggaggaattaaccatggttagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggactatgcatatccttagcgaaagctaaggattttttttgtctccccatgcgagagtaggg

SEQ ID NO: 75 - DNA, 1038 bp, Synthetic construct
  source 1..1038: mol_type = other DNA
tcgcaactctctactgtttctccatacccgttttttgggctacgctgaattcccggcagtttcccggtcccaagcccggataaagctcgagggggcgggaaaccgcctaagagcgcgaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaatggttagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgtttcgcgcagtaacactgccaatgccggtcccaagcccggataaaagtggagggctgcgcgaaacatgcatatccttagcgaaagctaaggattttttttgtctccccatgcgagagtaggg

SEQ ID NO: 76 - DNA, 2058 bp, Synthetic construct
  source 1..2058: mol_type = other DNA
cccgtgtaaaacgacggccagtttatctagtcagcttgattctagctgatcgtggaccggaaggtgagccagtgagttgattgcagtccagttacgctggagtctgaggctcgtcctgaatgatatgcggcctcggcggtagtggcggttctgctagcggtagaggcgggctgggtggccagggtgcaggtgcggctgcggctgccgcggcagcggccgcaggcggtgccggccaaggtggctatggcggcctgggttctcaggggactagtcaccatcatcatcaccatgcgcgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 77 - DNA, 1081 bp, Synthetic construct
  source 1..1081: mol_type = other DNA
gaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 78 - DNA, 1054 bp, Synthetic construct
  source 1..1054: mol_type = other DNA
gaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggcaaaggagatatacatatgcctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggttaattgaggcctgagtataaggtgacttatacttgtaatctatctaaacggggaacctctctagtagacaatcccgtgctaaattgtaggact

SEQ ID NO: 79 - DNA, 708 bp, Synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 80 - DNA, 27 bp, Synthetic construct
  source 1..27: mol_type = other DNA
ggaggaagcggtggcggtagtggcggg

SEQ ID NO: 81 - DNA, 27 bp, Synthetic construct
  source 1..27: mol_type = other DNA
ggtggcagcggcggagggagtggtgga

SEQ ID NO: 82 - protein, 36 aa, Synthetic construct
GRGGLGGQGAGAAAAAAAAAAGGAGQGGYGGLGSQG

SEQ ID NO: 83 - protein, 241 aa, Synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNRRRRR

SEQ ID NO: 84 - DNA, 36 bp, Synthetic construct
  source 1..36: mol_type = other DNA
caaggagatatacatatggctagcatgaatcacaaa

SEQ ID NO: 85 - DNA, 36 bp, Synthetic construct
  source 1..36: mol_type = other DNA
gttcctctatatgtataccgatcgtacttagtgttt

SEQ ID NO: 86 - DNA, 64 bp, Synthetic construct
  source 1..64: mol_type = other DNA
caccatgaaaacctgtattttcagggccaaggagatatacatatggctagcatgaatcacaaag

SEQ ID NO: 87 - DNA, 64 bp, Synthetic construct
  source 1..64: mol_type = other DNA
gtggtacttttggacataaaagtcccggttcctctatatgtataccgatcgtacttagtgtttc

SEQ ID NO: 88 - protein, 21 aa, Synthetic construct
HHENLYFQGQGDIHMASMNHK

SEQ ID NO: 89 - DNA, 106 bp, Synthetic construct
  source 1..106: mol_type = other DNA
taaaggaggaagcggtggcggtagtggcggggaaaacctgtattttcagggcggtggcagcggcggagggagtggtggacaaggagatatacatatggctagcatg

SEQ ID NO: 90 - DNA, 106 bp, Synthetic construct
  source 1..106: mol_type = other DNA
atttcctccttcgccaccgccatcaccgccccttttggacataaaagtcccgccaccgtcgccgcctccctcaccacctgttcctctatatgtataccgatcgtac

SEQ ID NO: 91 - protein, 35 aa, Synthetic construct
KGGSGGGSGGENLYFQGGGSGGGSGGQGDIHMASM

SEQ ID NO: 92 - RNA, 1073 nt, Synthetic construct
  source 1..1073: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgttttcttgggttaattgaggcctgagtataaggtgacttatacttgtaatctatctaaacggggaacctctctagtagacaatcccgtgctaaattgtaggact

SEQ ID NO: 93 - RNA, 1070 nt, Synthetic construct
  source 1..1070: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtttcttgggttaattgaggcctgagtataaggtgacttatacttgtaatctatctaaacggggaacctctctagtagacaatcccgtgctaaattgtaggact

SEQ ID NO: 94 - RNA, 1061 nt, Synthetic construct
  source 1..1061: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggttaattgaggcctgagtataaggtgacttatacttgtaatctatctaaacggggaacctctctagtagacaatcccgtgctaaattgtaggact

SEQ ID NO: 95 - RNA, 1073 nt, Synthetic construct
  source 1..1073: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaataaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 96 - RNA, 1070 nt, Synthetic construct
  source 1..1070: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 97 - RNA, 1067 nt, Synthetic construct
  source 1..1067: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 98 - RNA, 1064 nt, Synthetic construct
  source 1..1064: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 99 - RNA, 1061 nt, Synthetic construct
  source 1..1061: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 100 - RNA, 1049 nt, Synthetic construct
  source 1..1049: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggttaattgaggccggtgtataaggtgacttatacttgtaatctatctaaacggggaacctctctagtagacaatcccgtgctaaattgtaggact

SEQ ID NO: 101 - RNA, 1100 nt, Synthetic construct
  source 1..1100: mol_type = other RNA
ggccggccggccggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggactggccggccggcc

SEQ ID NO: 102 - RNA, 1076 nt, Synthetic construct
  source 1..1076: mol_type = other RNA
ggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccatgaaaacctgtattttcagggccaaggagatatacatatggctagcatgaatcacaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtgatgttttcttgggttaattgaggcctgagtataaggtgacttatacttgtaatctatctaaacggggaacctctctagtagacaatcccgtgctaaattgtaggact

SEQ ID NO: 103 - RNA, 1061 nt, Synthetic construct; identical to SEQ ID NO: 94
  source 1..1061: mol_type = other RNA